SEQUENCE LISTING (7 sequences)

SEQ ID NO: 1 - protein, 10 aa, Artificial Sequence
  synthetic construct
HIWLIYLWIV

SEQ ID NO: 2 - protein, 16 aa, Drosophila melanogater
RQIKIWFQNRRMKWKK

SEQ ID NO: 3 - protein, 27 aa, Artificial Sequence
  synthetic construct
GWTLNSAGYLLGKINLKALAALAKKIL

SEQ ID NO: 4 - protein, 25 aa, Human immunodeficiency virus
CFITKALGISYGRKKRRQRRRPPQC

SEQ ID NO: 5 - protein, 15 aa, Artificial Sequence
  variant of Seq ID No. 4
CGRKKRRQRRRPPQC

SEQ ID NO: 6 - protein, 19 aa, Artificial Sequence
  variant of Seq Id No. 4
CLGISYGRKKRRQRRPPQC

SEQ ID NO: 7 - protein, 37 aa, Artificial Sequence
  variant of Seq Id No. 4
GCFITKALGISYGRKKRRQRRRPPQGSQTHQVSLSKQ

(19) United States
(12) Patent Application Publication
(10) Pub. No.: US 2008/0160594 A1
(43) Pub. Date: Jul. 3, 2008

(54) Methods of Light Activated Release of Ligands from Endosomes

(75) Inventor: Tod M. Woolf, Sudbury, MA (US)

Correspondence Address: INVITROGEN CORPORATION;C/O INTELLEVATE, P.O. BOX 52050, MINNEAPOLIS, MN, 55402, US

(73) Assignee: INVITROGEN CORPORATION, Carlsbad, CA (US)

(21) Appl. No.: 11/556,149

(22) Filed: Nov. 2, 2006

Related U.S. Application Data
(63) Continuation of application No. 10/071,512, filed on Feb. 8, 2002, now abandoned.
(60) Provisional application No. 60/267,272, filed on Feb. 8, 2001.

(51) Int. Cl. C12N 13/00

(52) U.S. Cl. 435/173.1

(57) ABSTRACT

Methods for delivering ligands to a cell by using light to activate fluorescent ligands causing their release from endosomes. The instant methods thus increasing the efficiency of ligands, e.g. in vitro or at localized sites within a subject. The invention provides for the release of ligands by shining a light source on a cell to promote release of ligands into the cell where they can effect their function.

### RELATED APPLICATIONS

This application claims the priority of U.S. Provisional Patent Application No. 60/267,272, filed Feb. 8, 2001.

### BACKGROUND OF THE INVENTION

For antisense oligonucleotides to regulate gene expression, they must penetrate the cell membrane. Because oligonucleotides are anionic, they cannot passively diffuse through cell membranes. Oligonucleotides are believed to enter cells through two different active transport processes: adsorbtive endocytosis and fluid phase endocytosis (pinocytosis).

Adsorbtive endocytosis requires that the oligonucleotide adsorb to the surface of the cell. Charged oligonucleotides adsorb better to the surface of the cell than uncharged oligonucleotides and, therefore, are internalized better. Cell surface heparin-binding proteins facilitate the adsorbtion process.

Pinocytosis is the process where cells constitutively engulf water and dissolved solutes from the fluid phase. Pinocytosis is especially important for the internalization of oligonucleotides when the bulk-phase oligonucleotide concentration exceeds oligonucleotide/protein binding constants.

Virtually all internalized oligonucleotides as well as other molecules and particles that enter cells via the same pathway (e.g. peptides) end up in endosomes. Further, the leakage rate of oligonucleotides from endosomes to the cytoplasm is extremely slow. Stein. 1999. Biochimica et Biophysica Acta. 1489:45. Endosomes carry out several important processes associated with endocytosis including the sorting of internalized molecules and acidification. Acidification of the endosome is caused by an ATP-dependent proton pump and results in the endosome maturing to a lysosome. The oligonucleotides inside the lysosomes are completely broken down by lysosomal hydrolases. Liang et al. 1999. Pharmazie 54:8.

Various methods have been devised to help oligonucleotides bypass the endosomal compartments. Liang et al. 1999. Pharmazie 54:8. Vlassov. 1994. Biochimica et Biophysica Acta 1197:95. One way to avoid the endosomal barrier is to insert the oligonucleotides directly into the nucleus through electroporation or microinjection. This methodology, however, is invasive and is only appropriate for cells in vitro and cannot be readily applied clinically to in vivo tissue samples.

A second way to avoid the endosomal barrier is to incubate or couple oligonucleotides to viral peptides. For example, peptides derived from the haemagglutinin envelop protein of the Influenza virus are able to form a transmembrane channel through a conformational change induced by the acidification resulting from endocytosis. In another example, viral peptides from the Sendai virus (hemagglutiniating virus of Japan (HVJ)) can be attached to the surface of liposomes and cause the liposomes to fuse with the cell membrane and thereby avoid the endocytotic pathway. However, viral peptides are expensive to produce and can be immunogenic.

A third way to avoid the endosomal barrier is to form a liposome containing fusogenic and pH-sensitive lipids. Fusogenic lipids include phosphatidylethanolamine (PE) derivatives. pH-sensitive lipids contain titratable carboxylic acids such as cholesteryl hemisuccinate and oleic acid. At an alkaline pH, the liposome will retain its bilayer vesicle structure. When the pH decreases from the acidification of the endosome, the titratable head group of the pH-sensitive lipid is protonated causing the liposome to collapse. As the pH-sensitive lipids destabilize the bilayer structure, the fusogenic lipids promote membrane fusion between the liposome and the endosome causing oligonucleotides to be released out of the endosomes. Vlassov. 1994. Biochimica et Biophysica Acta 1197:95. Liposomes containing fusogenic and pH-sensitive lipids are unsatisfactory because they have a low capacity to entrap oligonucleotides.

A fourth way to avoid the endosomal barrier is through the use of biodegradable pH-sensitive surfactants. This method uses detergents that disrupt the phospholipid bilayers of the endosomes without disrupting the phospholipid bilayers of the cell membrane. In order to provide selectivity, a lysosomotropic amine (pKa 5-7) bearing a hydrophobic tail group is classified as a lysosomo-tropic detergent and forms the basis of biodegradable pH-sensitive surfactants (BPS). At alkaline pH, BPS are predominated by a hydrophobic tail and reside within lipid bilayers due to its limited surface-active properties. As the pH decreases in the endosome, the BPS will be protonated and will activate the membrane destabilization process using the surfactant-like properties of the ionized BPS resulting in the release of oligonucleotides from the endosome. BPS are problematic because if they are not completely degraded before the endosomes mature into lysosomes or are unprotonated until the lysosome stage, then the ionized BPS would disrupt the lysosomes and cause digestive enzyme release that could kill cells.

Therefore, the development of novel methods of releasing oligonucelotides and other molecules and particles from endosomes, thus enhancing their availability in a cell would be of great benefit.

### SUMMARY OF THE INVENTION

This invention advances the state of the prior art by providing novel methods of enhancing the availability of ligands inside a cell. Such methods are useful both in vitro and in vivo. In one aspect, the invention pertains to a method of delivering a ligand to a cell by contacting a cell with a ligand and a fluorophore; and illuminating the cell with a light that activates the fluorophore such that the ligand is delivered to the cell.

In one embodiment, the ligand is an oligonucleotide. In another embodiment, the ligand is peptide. In another embodiment, the ligand is a fluorescent virus. In still another embodiment, the ligand is a morpholino oligonucleotide. In still another embodiment, the ligand is a sense oligonucleotide. In yet another embodiment the ligand is an antisense oligonucleotide.

In one embodiment, the ligand enters an endosome of the cell during step (a). In another embodiment, the illuminating of step (b) causes the endosome containing the ligand to release the ligand.

In one embodiment, the light has a wavelength of between about 10 to about 380 nm. In another embodiment, the light has a wavelength of about 380 to about 500 nm.

In one embodiment, the cells are illuminated for less than about 2 minutes. In yet another embodiment, the cells are illuminated for less than about 1 minute.

In one embodiment, light is produced from a flexible endoscopic light source.

In another embodiment, the fluorophore and the ligand are linked via a covalent linkage.

In still another embodiment, the fluorophore is a fluorescein fluorophore.

In one embodiment, the fluorophore and the ligand contacted with the cell simultaneously.

In another aspect, the invention pertains to a method of delivering ligands to a cell by exposing a cell to a medium containing ligands and fluorophores wherein the ligands and fluorophores are not covalently linked; and illuminating the cell with a light that activates the fluorophores such that the ligands are delivered to a cell.

In another aspect the invention pertains to a method of releasing ligands from endosomes in cells present at a localized site in a subject comprising illuminating the cells at a localized site in the subject with a light such that the ligands are released at a localized site in the subject.

In one embodiment, the ligands are fluorescent oligonucleotides. In another embodiment, the ligands are fluorescent peptides. In another embodiment, the ligands are fluorescent viruses. In one embodiment, the ligands are fluorescent morpholino oligonucleotides.

In one embodiment, the fluorescent oligonucleotides are present in step (a) at a concentration of over 300 μM. In another embodiment, the fluorescent oligonucleotides are present in step (a) at a concentration of over 500 μM.

In one embodiment, the light has a wavelength that is about 10 to about 380 nm. In another embodiment, the light has a wavelength that is about 380 to about 500 nm.

In one embodiment, the cells are illuminated for less than 2 minutes. In another embodiment, the cells are illuminated for less than 1 minute.

In one embodiment, the light is produced from a flexible endoscopic light source.

In one embodiment, the ligands are covalently linked to a fluorescein fluorophore.

In one embodiment, the localized site is inside the mouth of the subject. In one embodiment, the localized site is inside the colon of the subject. In one embodiment, the localized site is on the skin of the subject. In another embodiment, the localized site is a tumor.

In another aspect, the invention pertains to a method of modulating protein production at a localized site in a subject by a group of cells of the subject to a ligand and a fluorophore; and illuminating the cells at a localized site in the subject with light that activates the fluorophore such that protein production at a localized site in the subject is modulated.

In one embodiment, protein production is enhanced. In another embodiment, protein production is inhibited.

In one aspect, the invention pertains to a method of modulating protein activity at a localized site in a subject by exposing a group of cells of the subject to a ligand and a fluorophore; and illuminating the cells at a localized site in the subject with light such that the fluorophore is activated and protein activity at a localized site in a subject is modulated.

In one embodiment, protein activity is enhanced. In another embodiment, protein activity is inhibited.

In one aspect, the invention pertains to a method of treating a disorder that would benefit from enhanced availability of a ligand in a cell by exposing a group of cells of the subject ligand and a fluorophore; and illuminating the cells with light that activates fluorophore, thereby enhancing the availability of the ligand and treating a disorder that would benefit from enhanced availability of the ligand.

### DRAWINGS

FIG. 1 shows A549 cells in the presence of oligonucleotides at 20× magnification under phase contrast microscopy

FIG. 2 shows A549 cells in the presence of oligonucleotides at 20> magnification under fluorescence microscopy

FIG. 3 shows Human Umbilical Vein Endothelial Cells (HUVECs) in the presence of oligonucleotides at 20× magnification under phase contrast microscopy.

FIG. 4 shows Human Umbilical Vein Endothelial Cells (HUVECs) in the presence of oligonucleotides at 20× magnification under fluorescence microscopy.

### DETAILED DESCRIPTION OF THE INVENTION

The instant methods are useful in increasing the availability of ligands to cells. As used herein, the term “ligand” includes molecules that enter cells by receptor mediated endocytosis, e.g., oligonucleotides, peptides, and other molecules and particles. Examples of ligands that enter cells by receptor mediated endocytosis include: (1) toxins and lectins such as diptheria toxin, pseudomonas toxin, cholera toxin, ricin, concanavalin A; (2) viruses such as rous sarcoma virus, semliki forest virus, vesicular stomatitis virus, and adenovirus; (3) serum transport proteins and antibodies such as transferrin, low density lipoprotein, transcobalamin, yolk proteins, IgE, polymeric IgA, maternal IgG, IgG via Fc receptors; (4) hormones and growth factors such as insulin, epidermal growth factor, growth hormone, thyroid stimulating hormone, nerve growth factor, calcitonin, glucagon, prolactin, luteinizing hormone, thyroid hormone, platelet derived growth factor, interferon, and catecholamines, as well as (5) other molecules that are bound to cell surface receptors that are recycled via this pathway.

Polypeptides and Peptides

As used herein, “polypeptide(s)” includes any peptide or protein comprising two or more amino acids joined to each other by peptide bonds or modified peptide bonds. “Polypeptide(s)” include both short chains, commonly referred to as peptides, oligopeptides and oligomers and longer chains generally referred to as proteins. Polypeptides may contain amino acids other than the 20 gene encoded amino acids. “Polypeptide(s)” include those modified either by natural processes, such as processing and other post-translational modifications, but also by chemical modification techniques. Such modifications are well described in basic texts and in more detailed monographs, as well as in research literature, and they are well known to those of skill in the art. It will be appreciated that the same type of modification may be present in the same or varying degree at several sites in a given polypeptide. Also, a given polypeptide may contain many types of modifications. Modifications can occur anywhere in a polypeptide, including the peptide backbone, the amino acid side-chains, and the amino or carboxyl termini. Modifications include, for example, acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphatidylinositol, cross-linking, cyclization, disulfide bond formation, demethylation, formation of covalent cross-links, formation of cysteine, formation of pyroglutamate, formylation, gamma-carboxylation, glycosylation, GPI anchor formation, hydroxylation, iodination, methylation, myristoylation, oxidation, proteolytic processing, phosphorylation, prenylation, racemization, glycosylation, lipid attachment, sulfation, gamma-carboxylation of glutamic acid residues, hydroxylation and ADP-ribosylation, selenoylation, sulfation, transfer-RNA mediated addition of amino acids to proteins, such as arginylation, and ubiquitination. See, for instance, Proteins—Structure And Molecular Properties, 2^{nd} Ed., T. E. Creighton, W. H. Freeman and Company, New York (1993) and Wold, F., Posttranslational Protein Modifications: Perspectives and Prospects, pgs. 1-12 in Posttranslational Covalent Modification Of Proteins, B. C. Johnson, Ed., Academic Press, New York (1983); Seifter et al. (1990) Meth. Enzymol. 182:626-646 and Rattan et al. (1992) Protein Synthesis: Posttranslational Modifications and Aging, Ann. N.Y. Acad. Sci. 663:48-62. Polypeptides may be branched or cyclic, with or without branching. Cyclic, branched and branched circular polypeptides may result from post-translational natural processes and may be made by entirely synthetic methods as well. The subject methods can be used to increase the intracellular availability of polypeptides, as well as smaller peptide molecules.

As used herein, the terms “isolated polypeptide” or “isolated protein” include a polypeptide or protein that is substantially free of other polypeptides, proteins, cellular material and culture medium when isolated from cells or produced by recombinant DNA techniques, or chemical precursors or other chemicals when chemically synthesized. An “isolated” or “purified” polypeptide or biologically active portion thereof is substantially free of cellular material or other contaminating polypeptides from the cell or tissue source from which the particular polypeptide is derived, or substantially free from chemical precursors or other chemicals when chemically synthesized. The language “substantially free of cellular material” includes preparations of a particular polypeptide in which the polypeptide is separated from cellular components of the cells from which it is isolated or recombinantly produced. In one embodiment, the language “substantially free of cellular material” includes preparations of a particular polypeptide having less than about 30% (by dry weight) of contaminating material, more preferably less than about 20% of contaminating material, still more preferably less than about 10% of contaminating material, and most preferably less than about 5% contaminating material. When a particular polypeptide or biologically active portion thereof is recombinantly produced, it is also preferably substantially free of culture medium, i.e., culture medium represents less than about 20%, more preferably less than about 10%, and most preferably less than about 5% of the volume of the polypeptide preparation.

The language “substantially free of chemical precursors or other chemicals” includes preparations of a particular polypeptide in which the polypeptide is separated from chemical precursors or other chemicals that are involved in the synthesis of the polypeptide. In one embodiment, the language “substantially free of chemical precursors or other chemicals” includes preparations of a particular polypeptide having less than about 30% (by dry weight) of chemical precursors or undesired chemicals, more preferably less than about 20% chemical precursors or undesired chemicals, still more preferably less than about 10% chemical precursors or undesired chemicals, and most preferably less than about 5% chemical precursors or undesired chemicals.

Peptides for use with the invention can be naturally occurring. As used herein, a “naturally-occurring” molecule refers to a particular molecule having a nucleotide sequence that occurs in nature. In addition, the invention includes naturally or non-naturally occurring variants of these polypeptides and nucleic acid molecules which retain the same functional activity. Such variants can be made, e.g., by mutation using techniques that are known in the art. Alternatively, variants can be chemically synthesized.

As used herein the term “variant(s)” includes polypeptides that differ in sequence from a reference polypeptide, but retains its essential properties.

Peptide mimetics that are structurally similar to therapeutically useful peptides may be used to produce an equivalent therapeutic or prophylactic effect. Generally, peptidomimetics are structurally similar to a paradigm polypeptide (i.e., a polypeptide that has a biological or pharmacological activity), but have one or more peptide linkages optionally replaced by a linkage selected from the group consisting of: —CH₂NH—, —CH₂S—, —CH₂—CH₂—, —CH═CH— (cis and trans), —COCH₂—, —CH(OH)CH₂—, and —CH₂SO—, by methods known in the art and further described in the following references: Spatola, A. F. in “Chemistry and Biochemistry of Amino Acids, Peptides, and Proteins,” B. Weinstein, eds., Marcel Dekker, New York, p. 267 (1983); Spatola, A. F., Vega Data (March 1983), Vol. 1, Issue 3, “Peptide Backbone Modifications” (general review); Morley, J. S. (1980) Trends Pharm. Sci. pp. 463-468 (general review); Hudson, D. et al. (1979) Int. J. Pept. Prot. Res. 14:177-185 (—CH2NH—, CH2CH2-); Spatola, A. F. et al. (1986) Life Sci. 38:1243-1249 (—CH2-S); Hann, M. M. (1982) J. Chem. Soc. Perkin Trans. I. 307-314 (—CH—CH—, cis and trans); Almquist, R. G. et al. (1980) J. Med. Chem. 23:1392-1398 (—COCH2-); Jennings-White, C. et al. (1982) Tetrahedron Lett. 23:2533 (—COCH2-); Szelke, M. et al. European Appln. EP 45665 (1982) CA: 97:39405 (1982) (—CH(OH)CH2-); Holladay, M. W. et al. (1983) Tetrahedron Lett. 24:4401-4404 (—C(OH)CH2-); and Hruby, V. J. (1982) Life Sci. 31:189-199 (—CH2-S—); each of which is incorporated herein by reference. A particularly preferred non-peptide linkage is —CH2NH—.

Such peptide mimetics may have significant advantages over polypeptide embodiments, including, for example: more economical production, greater chemical stability, enhanced pharmacological properties (e.g., half-life, absorption, potency, efficacy, etc.), altered specificity (e.g., a broad-spectrum of biological activities), reduced antigenicity, and others. Labeling of peptidomimetics usually involves covalent attachment of one or more labels, directly or through a spacer (e.g., an amide group), to non-interfering position(s) on the peptidomimetic that are predicted by quantitative structure-activity data and/or molecular modeling. Such non-interfering positions generally are positions that do not form direct contacts with the macromolecules(s) to which the peptidomimetic binds to produce the therapeutic effect. Derivitization (e.g., labeling) of peptidomimetics should not substantially interfere with the desired biological or pharmacological activity of the peptidomimetic.

Systematic substitution of one or more amino acids of an amino acid sequence with a D-amino acid of the same type (e.g., D-lysine in place of L-lysine) may be used to generate more stable peptides. In addition, constrained peptides may be generated by methods known in the art (Rizo and Gierasch (1992) Annu. Rev. Biochem. 61:387, incorporated herein by reference); for example, by adding internal cysteine residues capable of forming intramolecular disulfide bridges which cyclize the peptide.

Those of skill in the art can, without undue experimentation, produce polypeptides corresponding to particular peptide sequences. Such polypeptides may be produced in prokaryotic or eukaryotic host cells by expression of polynucleotides encoding the particular peptide sequence, frequently as part of a larger polypeptide. Alternatively, such peptides may be synthesized by chemical methods. Methods for expression of heterologous polypeptides in recombinant hosts, chemical synthesis of polypeptides, and in vitro translation are well known in the art and are described further in Maniatis et al., Molecular Cloning: A Laboratory Manual (1989), 2nd Ed., Cold Spring Harbor, N.Y.; Berger and Kimmel, Methods in Enzymology, Volume 152, Guide to Molecular Cloning Techniques (1987), Academic Press, Inc., San Diego, Calif.; Merrifield, J. (1969) J. Am. Chem. Soc. 91:501; Chaiken, I. M. (1981) CRC Crit. Rev. Biochem. 11:255; Kaiser et al. (1989) Science 243:187; Merrifield, B. (1986) Science 232:342; Kent, S. B. H. (1988) Ann. Rev. Biochem. 57:957; and Offord, R. E. (1980) Semisynthetic Proteins, Wiley Publishing, which are incorporated herein by reference).

Peptides can be produced, e.g., by direct chemical synthesis. Peptides can be produced as modified peptides, with nonpeptide moieties attached by covalent linkage to the N-terminus and/or C-terminus. In certain preferred embodiments, either the carboxy-terminus or the amino-terminus, or both, are chemically modified. The most common modifications of the terminal amino and carboxyl groups are acetylation and amidation, respectively. Amino-terminal modifications such as acylation (e.g., acetylation) or alkylation (e.g., methylation) and carboxy-terminal-modifications such as amidation, as well as other terminal modifications, including cyclization, may be incorporated into various embodiments of the invention. Certain amino-terminal and/or carboxy-terminal modifications and/or peptide extensions to the core sequence can provide advantageous physical, chemical, biochemical, and pharmacological properties, such as: enhanced stability, increased potency and/or efficacy, resistance to serum proteases, desirable pharmacokinetic properties, and others.

Larger subregions or fragments of the genes encoding a particular protein can be expressed as peptides by synthesizing the relevant piece of DNA using the polymerase chain reaction (PCR) (Sambrook, Fritsch and Maniatis, 2 Molecular Cloning; A Laboratory Manual, Cold Spring Harbor, N.Y., (1989)), and ligating the thus obtained DNA into an appropriate expression vector. Using PCR, specific sequences of the cloned double stranded DNA are generated, cloned into an expression vector, and then assayed.

Preferably, a chimeric or fusion protein is produced by standard recombinant DNA techniques. For example, DNA fragments coding for the different polypeptide sequences are ligated together in-frame in accordance with conventional techniques, for example by employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers which give rise to complementary overhangs between two consecutive gene fragments which can subsequently be annealed and reamplified to generate a chimeric gene sequence (see, for example, Current Protocols in Molecular Biology, eds. Ausubel et al. John Wiley & Sons: 1992). Moreover, many expression vectors are commercially available that already encode a fusion moiety (e.g., a GST polypeptide). A nucleic acid molecule encoding a particular peptide can be cloned into such an expression vector such that the fusion moiety is linked in-frame to the particular peptide. For example, hexa-histidine an be added to the peptide for purification by immobilized metal ion affinity chromatography (Hochuli, E. et al. (1988) Bio/Technology 6:1321-1325). In addition, to facilitate isolation of a particular peptide free of irrelevant sequences, specific endoprotease cleavage sites can be introduced between the sequences of a fusion moiety and the peptide. It may be necessary to increase the solubility of a peptide by adding functional groups to the peptide, or by omitting hydrophobic regions of the peptide.

The techniques for assembling and expressing DNA encoding a particular peptide, e.g., synthesis of oligonucleotides, PCR, transforming cells, constructing vectors, expression systems, and the like are well established in the art.

### Oligonucleotides

The term “ligand” also included oligonucleotides. The term “oligomer” or “oligonucleotide” includes two or more nucleomonomers covalently coupled to each other by linkages or substitute linkages. An oligomer may comprise, for example, between a few (e.g. 7, 10, 12, 15) or a few hundred (e.g., 100 or 200) nucleomonomers. For example, an oligomer of the invention preferably comprises between about 10 and about 50 nucleomonomers, between about 15 and about 40, or between about 20 and about 30 nucleomonomers. More preferably, an oligomer comprises about 25 nucleomonomers. Oligomers may comprise, for example, oligonucleotides, oligonucleosides, polydeoxyribonucleotides (containing 2′-deoxy-D-ribose) or modified forms thereof, e.g., DNA, polyribonucleotides (containing D-ribose or modified forms thereof), RNA, or any other type of polynucleotide which is an N-glycoside or C-glycoside of a purine or pyrimidine base, or modified purine or pyrimidine base. The term oligomer includes compositions in which adjacent nucleomonomers are linked via phosphorothioate, amide and other linkages (e.g., Neilsen, P. E., et al. 1991. Science. 254:1497).

The term “nucleomonomer” includes bases covalently linked to a second moiety. Nucleomonomers include, for example, nucleosides and nucleotides. Nucleomonomers can be linked to form oligomers that bind to target nucleic acid sequences in a sequence specific manner. The term “second moiety” as used herein includes substituted and unsubstituted cycloalkyl moieties, e.g. cyclohexyl or cyclopentyl moieties, and substituted and unsubstituted heterocyclic moeities, e.g. 6-member morpholino moeities or, preferably, sugar moieties. Sugar moieties include natural sugars, e.g. monosaccharides (such as pentoses, e.g. ribose), modified sugars and sugar analogs. Possible modifications include, for example, replacement of one or more of the hydroxyl groups with a halogen, a heteroatom, an aliphatic group, or the functionalization of the group as an ether, an amine, a thiol, or the like. For example, modified sugars include D-ribose, 2′-O-alkyl, 2′-amino, 2′-S-alkyl, 2′-halo, 2′-O-methyl, 2′-fluoro, 2′-methyoxy, 2′-ethyoxy, 2′-methoxyethoxy, 2′-allyloxy (—OCH₂CH═CH₂), 2′-propargyl, 2′-propyl, ethynyl, ethenyl, propenyl, and cyano and the like. In one embodiment, the sugar moiety can be a hexose and incorporated into an oligomer as described (Augustyns, K., et al., Nucl. Acids. Res. 1992. 18:4711). Exemplary nucleomonomers can be found, e.g., in U.S. Pat. No. 5,849,902.

The term “base” includes the known purine and pyrimidine heterocyclic bases, deazapurines, and analogs (including heterocycl substituted analogs, e.g. aminoethyoxy phenoxazine), derivatives (e.g., 1-alkenyl-, 1-alkynyl-, heteroaromatic-, and 1-alkynyl derivatives) and tautomers thereof. Examples of purines include adenine, guanine, inosine, diaminopurine, and xanthine and analogs (e.g., 8-oxo-N⁶-methyladenine or 7-diazaxanthine) and derivatives thereof. Pyrimidines include, for example, thymine, uracil, and cytosine, and their analogs (e.g., 5-methylcytosine, 5-methyluracil, 5-(1-propynyl)uracil, 5-(1-propynyl)cytosine and 4,4-ethanocytosine). Other examples of suitable bases include non-purinyl and non-pyrimidinyl bases such as 2-aminopyridine and triazines.

The term “nucleoside” includes bases which are covalently attached to a sugar moiety, preferably ribose or deoxyribose. Examples of preferred nucleosides include ribonucleosides and deoxyribonucleosides. Nucleosides also include bases linked to amino acids and/or amino acid analogs which may comprise free carboxyl groups, free amino groups, or protecting groups. Suitable protecting groups are well known in the art (see: P. G. M. Wuts and T. W. Greene, “Protective Groups in Organic Synthesis”, 2^{nd} Ed., Wiley-Interscience, New York, 1999; J. F. W. McOmie (ed.), “Protective Groups in Organic Chemistry”, Plenum, New York, 1973).

The term “nucleotide” includes nucleosides which further comprise a phosphate group or a phosphate analog.

As used herein, the term “linkage” includes a naturally occurring, unmodified phosphodiester moiety (—O—P(O)(O)—O—) that covalently couples adjacent nucleomonomers. As used herein, the term “substitute linkage” includes any analog or derivative of the native phosphodiester group that covalently couples adjacent nucleomonomers. Substitute linkages include phosphodiester analogs, e.g., such as phosphorothioate, phosphorodithioate, and P-ethyoxyphosphodiester, P-ethoxyphosphodiester, P-alkyloxyphosphotriester, methylphosphonate, and nonphosphorus containing linkages, e.g., acetals and amides. Such substitute linkages are known in the art (e.g., Bjergarde et al. 1991. Nucleic Acids Res. 19:5843; Caruthers et al. 1991. Nucleosides Nucleotides. 10:47).

Oligomers of the invention comprise 3′ and 5′ termini. The 3′ and 5′ termini of an oligomer can be substantially protected from nucleases e.g., by modifying the 3′ and/or 5′ linkages (e.g., U.S. Pat. No. 5,849,902 and WO 98/13526.). For example, oligomers can be made resistant by the inclusion of a “blocking group.” The term “blocking group” as used herein refers to substituents (e.g., other than OH groups) that can be attached to oligomers or nucleomonomers, either as protecting groups or coupling groups for synthesis (e.g., hydrogen phosphonate, phosphoramidite, or PO₃²⁻). “Blocking groups” also include “end blocking groups” or “exonuclease blocking groups” which protect the 5′ and 3′ termini of the oligomer, including modified nucleotides and non-nucleotide exonuclease resistant structures. Exemplary end-blocking groups include cap structures (e.g., a 7-methylguanosine cap), inverted nucleomonomers, e.g., with 3′-3′ and/or 5′-5′ end inversions (see e.g., Ortiagao et al. 1992. Antisense Res. Dev. 2:129), methylphosphonate, phosphoramidite, non-nucleotide groups (e.g., non-nucleotide linkers, amino linkers, conjugates) and the like. The 3′ terminal nucleomonomer can comprise a modified sugar moiety. The 3′ terminal nucleomonomer comprises a 3′-O that can optionally be substituted by a blocking group that prevents 3′-exonuclease degradation of the oligonucleotide. For example, the 3′-hydroxyl is esterified to a nucleotide through a 3′→3′ internucleotide linkage. For example, the alkyloxy radical can be methoxy, ethoxy, or isopropoxy, and preferably, ethoxy. Optionally, the 3′→3′ linked nucleotide at the 3′ terminus can be linked by a substitute linkage. To reduce nuclease degradation, the 5′ most 3′→5′ linkage can be a modified linkage, e.g., a phosphorothioate or a P-alkyloxyphosphotriester linkage. Preferably, the two 5′ most 3′→5′ linkages can be modified linkages. Optionally, the 5′ terminal hydroxy moiety can be esterified with a phosphorus containing moiety, e.g., phosphate, phosphorothioate, or P-ethoxyphosphate.

The term “chimeric oligomer” includes oligomers which comprise different component parts or regions which impart a desired quality to the oligomer. For example, specific regions of the oligomer (i.e., segments of the oligomer comprising at least one nucleomonomer) can provide stability against endonucleases, stability against exonucleases, complementarity with the target sequence, RNase H recruitment and activation, or the like. Regions may be multifunctional, e.g., providing more than one quality to the oligomer, e.g., complementarity and stability or RNase activation and complementarity. In addition, those of skill in the art will recognize that there may be more than one region imparting the same quality to one oligomer. The term “chimeric oligomer” includes oligomers having an RNA-like and a DNA-like region.

In an embodiment, the oligomer of the invention can activate RNase H. The language “RNase H activating region” includes a region of an oligomer, e.g. a chimeric oligomer, that is capable of recruiting RNase H to cleave the target RNA strand to which the oligomer is binds. Typically, the RNase activating region contains a minimal core (of at least about 3-5, typically between about 3-12, more typically, between about 5-12, and more preferably between about 5-10 contiguous nucleomonomers) of DNA or DNA-like nucleomonomers. (See e.g., U.S. Pat. No. 5,849,902). More preferably, the RNase H activating region comprises about nine contiguous deoxyribose containing nucleomonomers. Preferably, the contiguous nucleomonomers are linked by a substitute linkage, e.g., a phosphorothioate linkage.

The language “non-activating region” includes a region of an oligomer, e.g. a chimeric oligomer, that does not recruit or activate RNase H. Preferably, a non-activating region does not comprise phosphorothioate DNA. In a preferred embodiment, oligomers of the invention comprise at least one non-activating region. A non-activating region can comprise between about 10 and about 30 nucleomonomers. The non-activating region can be stabilized against nucleases and/or can provide specificity for the target by being complementary to the target and forming hydrogen bonds with the target nucleic acid molecule, preferably MRNA molecule, which is to be bound by the oligomer.

Oligomers of the invention can be single stranded or double stranded. Oligomers of the invention can be sense (resulting in protein synthesis) or antisense oligomers (resulting in inhibition of protein synthesis). Sense oligomers are described in WO 99/14346. Any antisense oligomers known in the art are suitable for use in the claimed methods, including those that operate via steric interactions and those that operate by hybridization to a target nucleic acid molecule.

In preferred embodiments, oligomers comprise one or more regions which are complementary to and can bind to a target nucleic acid sequence, e.g., by Watson/Crick or Hoogsteen binding. In one embodiment, oligomers of the invention are substantially complementary to a target RNA sequence. In a preferred embodiment, the antisense oligomers of the invention are complementary to a target RNA sequence over at least about 80% of the length of the oligomer. In a more preferred embodiment, antisense oligomers of the invention are complementary to a target RNA sequence over at least about 90-95% of the length of the oligomer. In a more particularly preferred embodiment, antisense oligomers of the invention are complementary to a target RNA sequence over the entire length of the oligomer. The ability of an oligomer to bind to a target sequence is primarily a function of the bases in the oligomer. Accordingly, elements ordinarily found in oligomers, such as the furanose ring and/or the phosphodiester linkage can be replaced with any suitable functionally equivalent element. The term “oligomer” includes any structure that serves as a scaffold or support for the bases of the oligomer, where the scaffold permits binding to the target nucleic acid molecule in a sequence-dependent manner.

In one embodiment, the oligomer of the invention is a ribozyme.

In a preferred embodiment, oligomers of the invention are morpholino oligonucleotides. Morpholino oligonucleotides are non-ionic and function by an RNase H-independent mechanism. Each of the 4 genetic bases (Adenine, Cytosine, Guanine, and Thymine) of the morpholino oligonucleotides is linked to a 6-membered morpholine ring. Morpholino oligonucleotides are made by joining the 4 different subunit types by non-ionic phosphorodiamidate intersubunit linkages. An example of a 2 subunit morpholino oligonucleotide is shown below.

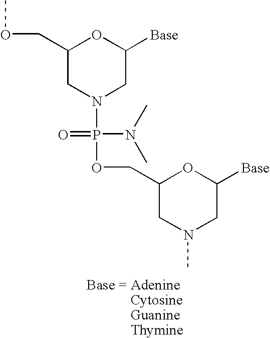

Morpholino oligonucleotides have many advantages over phosphorothioate oligonucleotides including: complete resistance to nucleases (Antisense & Nuc. Acid Drug Dev. 1996. 6:267); predictable targeting (Biochemica Biophysica Acta. 1999. 1489:141); reliable activity in cells (Antisense & Nuc. Acid Drug Dev. 1997. 7:63); excellent sequence specificity (Antisense & Nuc. Acid Drug Dev. 1997. 7:151); minimal non-antisense activity (Biochemica Biophysica Acta. 1999. 1489:141); and simple osmotic or scrape delivery (Antisense & Nuc. Acid Drug Dev. 1997. 7:291). Morpholino oligonucleotides are also preferred because of their non-toxicity at high doses. A discussion of the preparation of morpholino oligonucleotides can be found in Antisense & Nuc. Acid Drug Dev. 1997. 7:187.

Preferably, oligonucleotides used in the present invention are stabilized to be substantially resistant to endonuclease and exonuclease degradation

Uptake of Ligands Bells

To be taken up by cells ligands can delivered to, e.g., contacted with and taken up by one or more cells. The term “cells” refers to prokaryotic and eukaryotic cells, preferably vertebrate cells, and, more preferably, mammalian cells. In a preferred embodiment, ligands are contacted with human cells. Ligands can be contacted with cells in vitro or in vivo. Ligands are generally taken up by cells at a slow rate by endocytosis, but endocytosed ligands are generally sequestered in cells and not available to perform their function. The instant invention makes use of light to enhance the release of ligands from endosomes. In one embodiment, cellular uptake can be further facilitated, e.g., by electroporation or calcium phosphate precipitation. However, these procedures are only useful for in vitro or ex vivo embodiments, are not convenient and, in some cases, are associated with cell toxicity.

In another embodiment, delivery of ligands into cells can be further enhanced by suitable art recognized methods including calcium phosphate, DMSO, glycerol or dextran, electroporation, or by transfection, e.g., using cationic, anionic, and/or neutral lipid compositions or liposomes using methods known in the art (see e.g., WO 90/14074; WO 91/16024; WO 91/17424; U.S. Pat. No. 4,897,355; Bergan et al. 1993. Nucleic Acids Research. 21:3567). Enhanced delivery of ligands can also be mediated by the use of viruses, polyamine or polycation conjugates using compounds such as polylysine, protamine, or N1,N12-bis(ethyl)-spermine (see e.g., Bartzatt, R. et al. 1989. Biotechnol. Appl. Biochem. 11:133; Wagner E. et al. 1992. Proc. Natl. Acad. Sci. 88:4255)

### Conjugating Agents

Conjugating agents can be used to bind to a ligand in a covalent manner. In one embodiment, ligands can be derivatized or chemically modified to facilitate cellular uptake. For example, covalent linkage of a cholesterol moiety to an oligonucleotide can improve cellular uptake by 5- to 10-fold which in turn improves DNA binding by about 10-fold (Boutorin et al., 1989, FEBS Letters 254:129-132). Conjugation of octyl, dodecyl, and octadecyl residues enhances cellular uptake by 3-, 4-, and 10-fold as compared to unmodified ligands (Vlassov et al., 1994, Biochimica et Biophysica Acta 1197:95-108). Similarly, derivatization of ligands, e.g., oligomers, with poly-L-lysine can aid oligonucleotide uptake by cells (Schell, 1974, Biochem. Biophys. Acta 340:323, and Lemaitre et al., 1987, Proc. Natl. Acad. Sci. USA 84:648). Certain protein carriers can also facilitate cellular uptake of ligands, including, for example, serum albumin, nuclear proteins possessing signals for transport to the nucleus, and viral or bacterial proteins capable of cell membrane penetration. Therefore, protein carriers are useful when associated with or linked to the ligands. Accordingly, the present invention provides for derivatization of ligands with groups capable of facilitating cellular uptake, including hydrocarbons and non-polar groups, cholesterol, long chain alcohols (i.e. hexanol), poly-L-lysine and proteins, as well as other aryl or steroid groups and polycations having analogous beneficial effects, such as phenyl or naphthyl groups, quinoline, anthracene or phenanthracene groups, fatty acids, fatty alcohols and sesquiterpenes, diterpenes and steroids. A major advantage of using conjugating agents is to increase the initial membrane interaction that leads to a greater cellular accumulation of ligands. Methods for attaching such groups to ligands is known in the art. In another embodiment, ligands of the invention can be targeted to a particular receptor, e.g., one that is recycled by endocytosis.

### Encapsulating Agents

Encapsulating agents entrap ligands within vesicles. In another embodiment, an oligonucleotide may be associated with a carrier or vehicle, e.g., liposomes or micelles, although other carriers could be used, as would be appreciated by one skilled in the art. Liposomes are vesicles made of a lipid bilayer having a structure similar to biological membranes. Such carriers are used to facilitate the cellular uptake and/or targeting of the ligands, and/or improve the ligand's pharmacokinetic and/or toxicologic properties. For example, the ligands of the present invention may also be administered encapsulated in liposomes, pharmaceutical compositions wherein the active ingredient is contained either dispersed or variously present in corpuscles consisting of aqueous concentric layers adherent to lipidic layers. The ligands, depending upon solubility, may be present both in the aqueous layer and in the lipidic layer, or in what is generally termed a liposomic suspension. The hydrophobic layer, generally but not exclusively, comprises phopholipids such as lecithin and sphingomyelin, steroids such as cholesterol, more or less ionic surfactants such as diacetylphosphate, stearylamine, or phosphatidic acid, and/or other materials of a hydrophobic nature. The diameters of the liposomes generally range from about 15 nm to about 5 microns.

The use of liposomes as drug delivery vehicles offers several advantages. Liposomes increase intracellular stability, increase uptake efficiency and improve biological activity. Liposomes are hollow spherical vesicles composed of lipids arranged in a similar fashion as those lipids which make up the cell membrane. They have an internal aqueous space for entrapping water soluble compounds and range in size from 0.05 to several microns in diameter. Several studies have shown that liposomes can deliver nucleic acids to cells and that the nucleic acids remain biologically active. For example, a liposome delivery vehicle originally designed as a research tool, such as Lipofectin, can deliver intact nucleic acid molecules to cells.

Specific advantages of using liposomes include the following: they are non-toxic and biodegradable in composition; they display long circulation half-lives; and recognition molecules can be readily attached to their surface for targeting to tissues. Finally, cost-effective manufacture of liposome-based pharmaceuticals, either in a liquid suspension or lyophilized product, has demonstrated the viability of this technology as an acceptable drug delivery system.

### Complexing Agents

Complexing agents bind to the ligands by a strong (i.e. electrostatic) but non-covalent attraction. An example of a complexing agent includes cationic lipids. In one embodiment, cationic lipids can be used to deliver ligands to cells. The term “cationic lipid” includes lipids and synthetic lipids having both polar and non-polar domains and which are capable of being positively charged at or around physiological pH and which bind to polyanions, such as nucleic acids, and facilitate the delivery of ligands into cells. In general cationic lipids include saturated and unsaturated alkyl and alicyclic ethers and esters of amines, amides, or derivatives thereof. Straight-chain and branched alkyl and alkenyl groups of cationic lipids can contain, e.g., from 1 to about 25 carbon atoms. Preferred straight chain or branched alkyl or alkene groups have six or more carbon atoms. Alicyclic groups include cholesterol and other steroid groups. Cationic lipids can be prepared with a variety of counterions (anions) including, e.g., Cl⁻, Br⁻, I⁻, F⁻, acetate, trifluoroacetate, sulfate, nitrite, and nitrate. Examples of cationic lipids include poly(L-lysine), polyethylenimine, polyamidoamine (PAMAM) starburst dendrimers, avidin, Lipofectin (a combination of DOTMA and DOPE), Lipofectase, Lipofectamine, DOPE, Cytofectin (Gilead Sciences, Foster City, Calif.), and Eufectins (JBL, San Luis Obispo, Calif.). Examples of cationic liposomes include: N-[1-(2,3-dioleoloxy)-propyl]-N,N,N-trimethylammonium chloride (DOTMA), N-[1-(2,3-dioleoloxy)-propyl]-N,N,N-trimethylammonium methylsulfate (DOTAP), 3β-[N-(N′,N′-dimethylaminoethane)carbamoyl]cholesterol (DC-Chol), 2,3,-dioleyloxy-N-[2-(sperminecarboxamido)ethyl]-N,N-dimethyl-1-propanaminium trifluoroacetate (DOSPA), 1,2-dimyristyloxypropyl-3-dimethyl-hydroxyethyl ammonium bromide; and dimethyldioctadecylammonium bromide (DDAB). The cationic lipid N-(1-(2,3-dioleyloxy)propyl)-N,N,N-trimethylammonium chloride (DOTMA), for example, was found to increase 1000-fold the antisense effect of a phosphorothioate oligonucleotide. (Vlassov et al., 1994, Biochimica et Biophysica Acta 1197:95-108)

Cationic lipids have been used in the art to deliver oligonucleotides to cells (See e.g., U.S. Pat. Nos. 5,855,910; 5,851,548; 5,830,430; 5,780,053; 5,767,099; Lewis et al. 1996. Proc. Natl. Acad. Sci. USA 93:3176; Hope et al. 1998. Molecular Membrane Biology 15:1). Other lipid compositions which can be used to facilitate uptake of the instant ligands can be used in connection with the claimed methods. In addition to those listed supra, other lipid compositions are also known in the art and include, e.g., those taught in U.S. Pat. No. 4,235,871; and U.S. Pat. Nos. 4,501,728; 4,837,028; 4,737,323. In one embodiment lipid compositions can further comprise agents, e.g., viral proteins to enhance lipid-mediated transfections of oligonucleotides (Kamata et al. 1994. Nucl. Acids. Res. 22:536). In another embodiment, ligands are contacted with cells as part of a composition comprising an oligonucleotide, a peptide, and a lipid as taught, e.g., in U.S. Pat. No. 5,736,392. Improved lipids have also been described which are serum resistant (Lewis et al. 1996. Proc. Natl. Acad. Sci. 93:3176). Cationic lipids and other completing agents act to increase the number of oligonucleotides carried into the cell through endocytosis.

In another embodiment N-substituted glycine oligonucleotides (peptoids) can be used to optimize uptake of oligonucleotides. Peptoids have been used to create cationic lipid-like compounds for transfection (Murphy et al. 1998. Proc. Natl. Acad. Sci. 95:1517). Peptoids can be synthesized using standard methods (e.g., Zuckermann, R. N., et al. 1992. J. Am. Chem. Soc. 114:10646; Zuckermann, R. N., et al. 1992. Int. J. Peptide Protein Res. 40:497). Combinations of cationic lipids and peptoids, liptoids, can also be used to optimize uptake of the subject ligands (Hunag et al. 1998. Chemistry and Biology. 5:345). In one embodiment, liptoids can be synthesized by elaborating peptoid oligonucleotides and coupling the amino terminal submonomer to a lipid via its amino group (Hunag et al. 1998. Chemistry and Biology. 5:345).

It is known in the art that positively charged amino acids can be used for creating highly active cation lipids (Lewis et al. 1996. Proc. Natl. Acad. Sci. USA. 93:3176). In one embodiment, a composition for delivering ligands of the invention comprises a number of arginine, lysine, histadine and/or ornithine residues linked to a lipophilic moiety (see e.g., U.S. Pat. No. 5,777,153). In another, a composition for delivering ligands of the invention comprises a peptide having from between about one to about four basic residues. These basic residues can be located, e.g., on the amino terminal, c-terminal, or internal region of the peptide. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Apart from the basic amino acids, a majority or all of the other residues of the peptide can be selected from the non-basic amino acids, e.g., amino acids other than lysine, arginine, or histidine. Preferably a preponderance of neutral amino acids with long neutral side chains are used. For example, a peptide such as (N-term) His-Ile-Trp-Leu-Ile-Tyr-Leu-Trp-Ile-Val-(C-term) (SEQ ID NO: 1) could be used. In one embodiment such a composition can be mixed with the fusogenic lipid DOPE as is well known in the art.

In one embodiment, the cells to be contacted with an antisense construct are contacted with a mixture comprising the antisense construct and a mixture comprising a lipid, e.g., one of the lipids or lipid compositions described supra for between about 1 and about five days. In one embodiment, the cells are contacted with a mixture comprising a lipid and the antisense oligonucleotide for between about three days to as long as about 30 days. In another embodiment, a mixture comprising a lipid is left in contact with the cells for at least about five to about 20 days. In another embodiment, a mixture comprising a lipid is left in contact with the cells for at least about seven to about 15 days. In a preferred embodiment, a mixture comprising a lipid is left in contact with the cells for at least about three days. Surprisingly, given the low toxicity of the instant oligonucleotides, such prolonged incubation periods are possible.

For example, in one embodiment, an oligonucleotide can be contacted with cells in he presence of a lipid such as cytofectin CS or GSV (available from Glen Research; Sterling, Va.), GS3815, GS2888 for prolonged incubation periods as described herein.

In one embodiment the incubation of the cells with the mixture comprising a lipid and the antisense construct does not reduce the viability of the cells. Preferably, after the transfection period the cells are substantially viable. In one embodiment, after transfection, the cells are between at least about 70 and at least about 100 percent viable. In another embodiment, the cells are between at least about 80 and at least about 95% viable. In yet another embodiment, the cells are between at least about 85% and at least about 90% viable. Preferably, the cells are no less viable at the end of the incubation period with the mixture comprising the antisense construct and the lipid than similarly treated cells that are incubated with the same mixture for a period of only about 24 hours or less. Preferably, the prolonged transfection period is used to deliver the oligonucleotides of the instant invention to a cell.

In one embodiment, ligands are modified by attaching a peptide sequence that transports the oligonucleotide into a cell, referred to herein as a “transporting peptide.” In one embodiment, the composition includes an oligonucleotide which is complementary to a target nucleic acid molecule encoding the protein, and a covalently attached transporting peptide.

The language “transporting peptide” includes an amino acid sequence that facilitates the transport of a ligand into a cell. Exemplary peptides which facilitate the transport of the moieties to which they are linked into cells are known in the art, and include, e.g., HIV TAT transcription factor, lactoferrin, Herpes VP22 protein, and fibroblast growth factor 2 (Pooga et al. 1998. Nature Biotechnology. 16:857; and Derossi et al. 1998. Trends in Cell Biology. 8:84; Elliott and O'Hare. 1997. Cell 88:223).

For example, in one embodiment, the transporting peptide comprises an amino acid sequence derived from the antennapedia protein. Preferably, the peptide comprises amino acids 43-58 of the antennapedia protein (Arg-Gln-Ile-Lys-Ile-Trp-Phe-Gln-Asn-Arg-Arg-Met-Lys-Trp-Lys-Lys) (SEQ ID NO: 2) or a portion or variant thereof that facilitates transport of an oligonucleotide into a cell (see, e.g., WO 91/1898; Derossi et al. 1998. Trends Cell Biol. 8:84). Exemplary variants are shown in Derossi et al., supra.

In one embodiment, the transporting peptide comprises an amino acid sequence derived from the transportan, galanin (1-12)-Lys-mastoparan (1-14) amide, protein. (Pooga et al. 1998. Nature Biotechnology 16:857). Preferably, the peptide comprises the amino acids of the transportan protein shown in the sequence GWTLNSAGYLLGKINLKALAALAKKIL (SEQ ID NO: 3) or a portion or variant thereof that facilitates transport of an oligonucleotide into a cell.

In one embodiment, the transporting peptide comprises an amino acid sequence derived from the HIV TAT protein. Preferably, the peptide comprises amino acids 37-72 of the HIV TAT protein, e.g., shown in the sequence C(Acm)FITKALGISYGRKKRRQRRRPPQC (SEQ ID NO: 4) (TAT 37-60; where C(Acm) is Cys-acetamidomethyl) or a portion or variant thereof, e.g., C(Acm)GRKKRRQRRRPPQC (SEQ ID NO: 5) (TAT 48-40) or C(Acm)LGISYGRKKRRQRRPPQC (SEQ ID NO: 6) (TAT 43-60) that facilitates transport of an oligonucleotide into a cell (Vives et al. 1997. J. Biol. Chem. 272:16010). In another embodiment the peptide (G)CFITKALGISYGRKKRRQRRRPPQGSQTHQVSLSKQ (SEQ ID NO: 7) can be used.

Portions or variants of transporting peptides can be readily tested to determine whether they are equivalent to these peptide portions by comparing their activity to the activity of the native peptide, e.g., their ability to transport fluorescently labeled oligonucleotides to cells. Fragments or variants that retain the ability of the native transporting peptide to transport an oligonucleotide into a cell are functionally equivalent and can be substituted for the native peptides.

Ligands can be attached to the transporting peptide using known techniques, e.g., (Prochiantz, A. 1996. Curr. Opin. Neurobiol. 6:629; Derossi et al. 1998. Trends Cell Biol. 8:84; Troy et al. 1996. J. Neurosci. 16:253), Vives et al. 1997. J. Biol. Chem. 272:16010). For example, in one embodiment, oligonucleotides bearing an activated thiol group are linked via that thiol group to a cysteine present in a transport peptide (e.g., to the cysteine present in the β turn between the second and the third helix of the antennapedia homeodomain as taught, e.g., in Derossi et al. 1998. Trends Cell Biol. 8:84; Prochiantz. 1996. Current Opinion in Neurobiol. 6:629; Allinquant et al. 1995. J. Cell Biol. 128:919). In another embodiment, a Boc-Cys-(Npys)OH group can be coupled to the transport peptide as the last (N terminal) amino acid and an oligonucleotide bearing an SH group can be coupled to the peptide (Troy et al. 1996. J. Neurosci. 16:253). In one embodiment, a linking group can be attached to a nucleomonomer and the transporting peptide can be covalently attached to the linker. In one embodiment, a linker can function as both an attachment site for a transporting peptide and can provide stability against nucleases. Examples of suitable linkers include substituted or unsubstituted C₁-C₂₀ alkyl chains, C₁-C₂₀ alkenyl chains, C₁-C₂₀ alkynyl chains, peptides, and heteroatoms (e.g., S, O, NH, etc.). Other exemplary linkers include bifunctional crosslinking agents such as sulfosuccinimidyl-4-(maleimidophenyl)-butyrate (SMPB) (see e.g., Smith et al. Biochem J 1991. 276: 417-2).

In one embodiment, ligands of the invention are synthesized as molecular conjugates which utilize receptor-mediated endocytotic mechanisms for delivering genes into cells (See e.g., Bunnell et al. 1992. Somatic Cell and Molecular Genetics. 18:559 and the references cited therein).

Oligomer Synthesis

Oligomers of the invention can be synthesized by any methods known in the art, e.g., using enzymatic synthesis and chemical synthesis.

Preferably, chemical synthesis is used. Chemical synthesis of linear oligomers is well known in the art and can be achieved by solution or solid phase techniques. Preferably, synthesis is by solid phase methods. Oligomers can be made by any of several different synthetic procedures including the phosphoramidite, phosphite triester, H-phosphonate and phosphotriester methods, typically by automated synthesis methods. Oligomer synthesis protocols are well known in the art and can be found, e.g., in U.S. Pat. No. 5,830,653; WO 98/13526; Stec et al. 1984. J. Am. Chem. Soc. 106:6077; Stec et al. 1985. J. Org. Chem. 50:3908; Stec et al. J. Chromatog. 1985. 326:263; LaPlanche et al. 1986. Nuc. Acid. Res. 1986. 14:9081; Fasman G. D., 1989. Practical Handbook of Biochemistry and Molecular Biology. 1989. CRC Press, Boca Raton, Fla.; Lamone. 1993. Biochem. Soc. Trans. 21:1; U.S. Pat. No. 5,013,830; U.S. Pat. No. 5,214,135; U.S. Pat. No. 5,525,719; Kawasaki et al. 1993. J. Med. Chem. 36:831; WO 92/03568; U.S. Pat. No. 5,276,019; U.S. Pat. No. 5,264,423).

The synthesis method selected can depend on the length of the desired oligomer and such choice is within the skill of the ordinary artisan. For example, the phosphoramidite and phosphite triester method produce oligomers having 175 or more nucleotides while the H-phosphonate method works well for oligomers of less than 100 nucleotides. If modified bases are incorporated into the oligomer, and particularly if modified phosphodiester linkages are used, then the synthetic procedures are altered as needed according to known procedures. In this regard, Uhlmann et al. (1990, Chemical Reviews 90:543-584) provide references and outline procedures for making oligomers with modified bases and modified phosphodiester linkages. Other exemplary methods for making oligomers are taught in Sonveaux. 1994. “Protecting Groups in Oligonucleotide Synthesis”; Agrawal. Methods in Molecular Biology 26:1. Exemplary synthesis methods are also taught in “Oligonucleotide Synthesis—A Practical Approach” (Gait, M. J. IRL Press at Oxford University Press. 1984). Moreover, linear oligomers of defined sequence can be purchased commercially.

The oligomers may be purified by polyacrylamide gel electrophoresis, or by any of a number of chromatographic methods, including gel chromatography and high pressure liquid chromatography. To confirm a nucleotide sequence, oligomers may be subjected to DNA sequencing by any of the known procedures, including Maxam-Gilbert sequencing, Sanger sequencing, capillary electrophoresis sequencing the wandering spot sequencing procedure or by using selective chemical degradation of oligomers bound to Hybond paper. Sequences of short oligomers can also be analyzed by laser desorption mass spectroscopy or by fast atom bombardment (McNeal, et al., 1982, J. Am. Chem. Soc. 104:976; Viari, et al., 1987, Biomed. Environ. Mass Spectrom. 14:83; Grotjahn et al., 1982, Nuc. Acid Res. 10:4671). Sequencing methods are also available for RNA oligomers.

The quality of oligomers synthesized can be verified by testing the oligomer by capillary electrophoresis and denaturing strong anion HPLC (SAX-HPLC) using, e.g., the method of Bergot and Egan. 1992. J. Chrom. 599:35.

It will be understood that the oligomers of the invention can be synthesized to comprise one or more of the disclosed improvements. For example, in one embodiment, an oligomer of the invention comprises a nucleomonomer containing a propargyl group. In another embodiment, an oligomer of the invention comprises a nucleomonomer containing an affinity enhancing agent. In another exemplary embodiment, an oligomer of the invention comprises unmodified RNA nucleomonomers. In one embodiment, an oligomer of the invention comprises at least two of the above improvements. In one embodiment, an oligomer of the invention comprises at least three of the above improvements. One of skill in the art will recognize that given the teachings of the specification, multiple variations and combinations of these improved oligomers can be made.

### Dosage of Ligands

Ligands can be used at a local or systemic concentration sufficient to effect the result desired. As used herein, the local concentration of a ligand is the concentration at the site of the cells being contacted with the ligand. The local concentration of a ligand may be much greater than the concentration of the ligand at other sites or the systemic concentration. In one embodiment, ligands are used at a local concentration of at least about 0.1 μM, 1 μM, at least about 10 μM, or at least about 100 μM. Ligands can be administered using the claimed methods in a one-time-only fashion or can be administered repeatedly.

### Light for Activating Fluorescence

An appropriate wavelength of light used for activating the fluorescently labeled ligands or fluorophores can be obtained from any light source, e.g., a fluorescent lamp or an ordinary light bulb. Preferably, cells are illuminated with light at the time of or after being contacted with a ligand. A preferred light source is a laser or a light source having a lens that focuses the beam of light to a narrow area. It is preferred that the light used for activating the fluorescently labeled oligonucleotides or fluorophores be UV light (i.e. λ=10-380 nm) or blue light (i.e. λ=380-500 nm).

The light source may be powered in virtually any manner including an electrical outlet, battery power, and solar power. In a preferred embodiment, the light source is handheld and is powered by a battery.

In one embodiment, cells in a particular area of the body may be reached by a light source using a variation of endoscopy. For example, a light source may be linked to a flexible instrument that can be inserted through an opening of the body such as the mouth or rectum. In particular, the light source may comprise a lighted optical shaft or open tube. Preferably, the optical shaft used comprises bundles of fiber optic glass fibers that are bundled to together to form a flexible light source that can be easily bent and twisted around corners. Examples of endoscopic tools that can be adapted for use in the present invention include the bronchoscope (for examination of the bronchial tubes); gastroscope (for examination of the stomach); proctosigmoidoscope (for examination of the rectum and lower colon); and the cytoscope (for examination of the bladder).

An incision may be required to insert the light source into the subject. Examples of endoscopic tools requiring an incision that can be adapted for use in the present invention include the thoracoscope (for examination of the chest cavity and surface of the lungs through a small incision between the ribs); peritoneoscope (for examination of the abdominal cavity and lower surface of the liver and gallbladder through a small incision in the abdominal wall); and culdoscope (for examination of the female pelvic organs through a small vaginal incision).

Organs that can be visualized by the flexible light source of the present invention include the esophagus, stomach, lungs, bronchial tubes, duodenum, colon, liver, bladder, pancreas, and gall bladder. One skilled in the art would recognize other variations of the flexible light source used in the present invention as well as other organs that may be visualized.

In one embodiment, the light source may be controlled by a manually operated on/off switch. In an embodiment, the intensity of the light from the light source can be adjusted up or down. In a preferred embodiment, the light source is controlled by an automatic on/off switch that is controlled by a timer. In an embodiment, the timer is turned on and off by a computer. The light source may illuminate the cell(s) containing the oligonucleotides for an appropriate length of time to release oligomers from endosomes. This can be readily determined by using fluorescence microscopy to observe the movement of fluorescence out of endosomes. This can be done, for example, on the cell population being treated or on a test population comprising cells of the same type as those being treated. In one embodiment, the cells are contacted with an oligonucleotide for less than about 10 minutes, more preferably for less than about 5 minutes. In a preferred embodiment, the light source illuminates the cell(s) containing the oligonucleotides for less than about 2 minutes. In another preferred embodiment, the light source illuminates the cell(s) containing the oligonucleotides for less than about 1 minute. In another preferred embodiment, the light source illuminates the cell(s) containing the oligonucleotides for less than 30 seconds.

In addition, body fluids (i.e. blood, lymph) can be removed from the body, illuminated, and returned to the body. Strict aspectic technique should be used during these procedures to avoid introducing pathogens into the patients blood stream. For example, during leukophoresis, blood is drawn from the body, cleansed of white bloods cells, and returned to the body. It is possible to expose the blood drawn from the body during leukophoresis to light. Similarly, lymphocytes can be extracted from a one person's blood and transferred intravenously to a different person's blood. During this transfer, the lymph can be exposed to light. Also, during hemodialysis, a person's blood is passed through a kidney machine, usually for a period of 4 hours. During this time, the blood can be illuminated to promote the release of oligonucleotides and other molecules and particles from the endosomes of the cells in the blood. During hemodialysis, two needles are inserted in the subject's arm, one to take blood and another to return blood to the subject. To ensure repeated access to a person's blood stream, an artificial connection between an artery and a vein, an arteriovenous fistula, is made in the forearm. This result in dilation of the vein so it can be easily punctured with a needle each time dialysis is undertaken. Until a fistula has been inserted in the forearm, a temporary jugular catheter may be placed in the lower neck as another way to take the subject's blood.

### Fluorophores

The ligands of the present invention are brought into contact with cells in the presence of one or more fluorescent molecules (fluorophores), e.g., in the form of a mixture comprising a ligand and a fluorophore. Preferably, the ligands of the present invention have one or more fluorescent labels chemically bound, e.g., via a covalent linkage) to them. Fluorophores are substances that produce light (fluoresce) after being excited by radiant energy. Fluorescence occurs when electrons which were displaced to excited energy states by energy absorbed from radiation return to lower energy states. Electromagnetic energy is given off as the electrons return to lower energy states. Fluorescence begins when the fluorophore is irradiated and ends when the irradiation ends, with a short delay of around 0.1-10 ns. The intensity of fluorescence is usually proportional to the intensity of irradiation.

Fluorophores can be synthesized via the incorporation of commercially available fluorescently labeled phosphoramidites or by using a linker to a number of sites on an oligomeric compound. A wide variety of commercially available fluorophores exist which are suitable for use in the present invention including, but are not limited to eosin, fluorescamine, naphthalene derivatives (e.g. dansyl chloride), anthracene derivatives (e.g. N-hydroxysuccinimide ester of anthracene propionate), pyrene derivatives (erg. N-hydroxysuccinimide ester of pyrene butyrate), fluorescein derivatives (e.g. fluorescein isothiocyanate), rhodamine derivatives (e.g. rhodamine isothiocyanate, tetramethyl rhodamine, Lissamine Rhodamine B, carboxyrhodamine 6G, 5(6)-carboxytetramethylrhodamine (5(6)-TAMRA)), Lucifer Yellow, Lucifer Yellow VS 4-acetamido-4′-isothiocyanatostilbine-2,2-disulfonic acid, 7-diethylamine-3-(4′-isothiocyanatostilbine)-4-methylcoumarin, B-phycoerythrin, 9-acridineisocyanate derivatives, and succinimdyl-1-pyrenebutyrate, fluorescamine, OPA, NDA, ethidium bromide, acridine, JOE, C6-NBD, DIO-Cn-(3), BODIPY-FL, propidium iodide, dil-Cn-(3), texas red, Cy3, dil-Cn-(5), allophycocyanin, Cy5, 5-fluorescein (5-FITC); 6-carboxyfluorescein (6-FAM); 5(6)-carboxyfluorescein (5(6)-FAM); 6-hexachlorofluorescein (6-HEX); 6-tetrachlorofluorescein (6-TET); 6-JOE; Oregon Green® 488; Oregon Green 500; Oregon Green 514; BODIPY FL-X; BODIPY-TMR-X; BODIPY R6G; BODIPY 650/665; BODIPY 564/570; BODIPY 581/591; BODIPY TR-X; BODIPY 630/650; BODIPY 493/503; and FAM. Other fluorophore precursors are sold by Molecular Probes, Inc. Eugene, Oreg. In addition, other fluorophores are described in PCT application WO 92/03464. Examples of fluorophores can be found in Leeds et al, U.S. Pat. No. 6,127,124; Klock, Jr., U.S. Pat. No. 6,048,707; Ahlem, et al., U.S. Pat. No. 5,955,612; and Mathies, et al., U.S. Pat. No. 5,654,419. Methods for identifying fluorophores can be found in Gorfinkel, et al., U.S. Pat. No. 5,784,157. In another embodiment, a fluorophore of the present invention may be contacted with a cell (administered) concurrently with a ligand without being covalently linked to the ligand. For instance, the fluorophores may be attached to the ligands by a noncovalent linkage. An example of a noncovalent linkage is where the ligand is linked to biotin (or streptavidin) and the fluorophore is linked to streptavidin (or biotin) and the molecule and fluorophore are held together by the noncovalent interaction of streptavidin and biotin Alternatively, the fluorophore can be contacted with a cell in conjunction with the ligand without being chemically bound to the ligand. Preferably, the fluorophore is intermixed with the ligands with which it is administered.

Preferably, a ligand and a fluorophore are contacted with a cell such that upon exposure to light the availability of the ligand within the cell increases. Preferably, a ligand and a fluorophore are contacted with a cell such that upon exposure to light, the ligand is released from at least one endosome of the cell.

### Linking of Fluorophores to Ligands

Chemical bonding of fluorescent labels, with or without a linking or tethering group, to oligomeric compounds, is well known in the art (see for example: Hill, J. J. and Royer, C. A., Methods Enzymol., 1997, 278, 390-416; and Amann et al., Microbiol. Rev., 1997, 20, 191-200). Typically, the fluorescent label is attached via a covalent bond using a tethering moiety.

Exemplary linking or tethering moieties useful for attaching groups including fluorescent labels to ligands of the invention include N-(2-bromoethyl)phthalimide, -(3-bromopropyl)phthalimide and N-(4-bromobutyl)phthalimide (Aldrich Chemical Co., Inc., Milwaukee, Wis.). Other phthalimide-protected amine compounds can be conveniently synthesized from appropriate alkyl, aralkyl or aryl halides and phthalimide. Further representative compounds include N-(7-bromoheptyl)phthalimide; N-(8-bromooctyl)phthalimide; N-(9-bromononyl)phthalimide; N-(10-bromododecyl)phthalimide; N-(7-bromoundecyl)phthalmide; N-(12-bromodocecyl)phthalimide; N-(13-bromotridecyl)phthalimide; N-(14-bromotetradecyl)phthalimide; N-(15-bromopentadecyl)phthalimide; N-(16-bromo-hexadecyl)phthalimide; N-(17-bromoheptadecyl)phthalimide; N-(18-bromooctadecyl)phthalimide; N-(19-bromononadecyl)phthalimide; N-(3-bromo-2-methylpropyl)phthalimide; N-(4-bromo-2-methyl-3-ethylbutyl)phthalimide; N-(3-bromo-2,2-diethyl-propyl)phthalimide; N-(4-bromo-3-propylbutyl)phthalimide; N-(10-bromo-2,8-dibutyldecyl)phthalimide; N-(8-bromo-6,6-dimethyloctyl)phthalimide; N-(8-bromo-6-propyl-6-butyloctyl)phthalimide; N-(4-bromo-2-methylbutyl)phthalimide; N-(5-bromo-2-methylpentyl)phthalimide; N-(5-bromo-3-methylpentyl)phthalimide; N-(6-bromo-2-ethylhexyl)phthalimide; N-(5-bromo-3-penten-2-one)phthalimide; N-(4-bromo-3-methyl-2-butanol)phthalimide; N-(8-bromo-3-amino-4-chloro-2-cyanooctyl)phthalimide; N-(7-bromo-3-methoxy-4-heptanal)phthalimide; N-(4-bromo-2-iodo-3-nitrobutyl)phthalimide; N-(12-bromo-4-isopropoxydodecyl)phthalimide; N-(10-bromo-4-azido-2-nitrodecyl)phthalimide; N-(9-bromo-5-mercaptononyl)phthalimide; N-(5-bromo-4-aminopentenyl)phthalimide; N-(5-bromo-penten-2-yl)phthalimide; N-(3-bromoallyl)phthalimide; N-(4-bromocrotyl)phthalimide; N-(3-bromopropargyl)phthalimide; N-(1-bromonaphth-4-yl)phthalimide; N-(2-bromoanthrac-7-yl)-phthalimide; and N-(2-bromophenanthr-6-yl)phthalimide. Such halide compounds are then reacted with an appropriate 2, 6 or 8-oxygen, 2, 6 or 8-sulfur or 2, 6 or 8 amine substituted purine or purine containing nucleosides. In addition, as disclosed in U.S. Pat. No. 5,846,719, aminophosphate linkers (available from Applied Biosystems (Foster City, Calif.)) may be used. Other sites of reactivity are available on oligonucleotide analogs having non-naturally occurring sites thereon.

Terminal and internal labeling methods are also known in the art and may be used to link the fluorescent dyes at their respective sites to the oligonucleotides. Examples of 5′-terminal labeling methods include a) periodate oxidation of a 5′-to-5′-coupled ribonucleotide followed by reaction with an amine-containing label, b) condensation of ethylenediamine with a 5′-phosphorylated polynucleotide followed by reaction with an amine-reactive label, and c) introduction of an aliphatic amine substituent using an aminohexyl phosphite reagent in solid-phase DNA synthesis followed by reaction with an amine-reactive label. Labels may also be linked to synthetic DNA oligonucleotides at specific locations using special aliphatic amine-containing nucleotide phosphoramidite reagents. Selection of an appropriate method for linking the selected labels to the signal primer and performing the linking reactions are routine in the art.

In addition, the fluorophore can be attached to deoxynucleoside triphosphates (dNTPs) and those fluorophore-labeled deoxynucleoside triphosphates can in turn use to synthesize a fluorophore-labeled oligonucleotide using for example a DNA polymerase. See Goodman, et al., U.S. Pat. No. 5,945,312. By controlling the ratio of fluorophore-labeled dNTPs to unlabeled dNTPs the level of fluorescence of the resulting oligonucleotides can be controlled.

Fluorophores can be attached to peptides according to the methods described in Faure, et al., U.S. Pat. No. 6,054,557. In general, reactions between peptides and fluorophores are carried out by modifying amino acid functional groups, most typically a thiol or amine group, so that the moieties may be easily conjugated. Reactions for such modifications are described in the “Handbook of Fluorescent Probes and Research Chemicals—5th Edition” by Richard P. Haugland (1992), the contents of which are incorporated herein by reference. In general, thiols react with alkylating groups (R′-Z) to yield relatively stable thiol ethers (R—S—R′), with the leaving group Z preferably being a halogen (e.g., Cl, Br, or I) or a similar moiety. The most common reagents for derivatization of thiols are haloacetyl derivatives. Reaction of these reagents with thiols proceeds rapidly at or below room temperature in the physiological pH range.

Fluorophores may also be attached to amino acid amine groups. The conditions used to modify amine moieties of the desired peptide will depend on the class of amine (e.g., aromatic or aliphatic) and its basicity. Aliphatic amines, such as the α-amino group of lysine, are moderately basic and reactive with acylating reagents. The concentration of the free-base form of aliphatic amines below pH 8 is very low; thus, the kinetics of acylation reactions of amines by isothiocyanates, succinimidyl esters, and other reagents is strongly pH-dependent. Although amine acylation reactions should usually be carried out above pH 8.5, the acylation reagents degrade in the presence of water, with the rate increasing as the pH increases. The α-amino function of the amino terminus usually has a pKa of ˜7, thereby allowing it to be selectively modified by reaction at neutral pH.

In general, reactive groups on the fluorophore, such as unsaturated alkyl groups, will react with the modified amino acid. The chemical structure of the fluorophore may affect the synthetic route used to synthesize the compound. It may be necessary, for example, to modify the fluorophore so that it includes a reactive group prior to exposure to the desired peptide.

In certain peptides, the carboxy terminus is the only part of the molecule which can be attached to a fluorophore without disrupting the peptide's biological activity. In these cases, it is therefore necessary to add a separate “linker” group to the peptide. Since the N-hydroxysuccinimide esters (NHS) or isothiocyanate forms of fluorophores do not readily react with carboxylic groups or carboxyl amine groups, these groups must first be modified to a provide a functional site (e.g., a primary amino group) for conjugation with fluorophores. For example, fluorescent opioid peptides include linker groups to maintain their biological activity. In this case, an aminopentyl group is grafted onto the C-terminal amino acid by aminolysis of the opioid peptide with 1,5-diaminopentane as described below. Aminopentyl linker groups can also be added to a peptide when the peptide is incubated with carbodimides. Water soluble carbodimides are widely used for carboxyl-amine conjugation and may also serve to link fluorophores to the carboxy terminus of peptides.

Whether or not to include a linker group is usually determined empirically by testing a fluorescent peptide labeled at various amino acid sites and finding that it has lost biological activity. For some peptides, structure-activity studies show that the entire amino terminus and central portion of the peptide are involved in receptor binding. This suggests that only the carboxy terminus of the peptide can be modified without disrupting biological activity.

### Treatment of Disease or Disorders

By illuminating a localized site on a subject, it is possible to cause ligands to be made more available to cells. Using the subject methods, rather than being sequestered in endosomes, ligands are released from those endosomes into the intracellular milieu where they can perform their effector function. In one embodiment, such ligands can be made more available to cells which are at a specific location in a subject or to specific types of cells in the subject. For example, in one embodiment, cells in the mouth, the eyes, the inside of the ears, the skin, pharynx, trachea, esophagus, stomach, lungs, bronchial tubes, vagina, cervix, duodenum, colon, liver, bladder, pancreas, and gall are illuminated to release ligands from the endosomes. In another embodiment, a method of the invention is used to make ligands more available, e.g., to epithelial cells.

By releasing the ligands of the present invention at localized sites in a subject, various diseases can be treated, particularly diseases that have localized effects in the subject. For example, oligonucleotides that act in an antisense manner to negatively regulate one or more oncogenes (i.e., ras, SHP-1, MDM2) could be released at the location of a tumor by using the oligonucleotides and fluorophores of the present invention and shining light on the tumor, thereby causing the oligonucleotides to be released at the site of the tumor and activating in inhibit the expression of the oncogene(s). By inhibiting the expression of the oncogene(s), the oligonucleotides of the present invention can be used to treat cancer at the specific localized sites that most require treatment.

The oligonucleotides of the present invention can also be sense oligonucleotides. By releasing sense oligonucleotides at localized sites in a subject, diseases or disorder resulting from a deficiency of a protein can be treated. Examples of proteins that may be encoded by sense oligonucleotides of the present invention to treat diseases or disorders requiring the increased production of a polypeptide include: immunoregulatory proteins (e.g. a cytokine to enhance an immune response, an antigen to provoke an immune response), growth factors, tumor suppressors (e.g. p53), and molecules that down modulate an immune response (e.g. CTLA4).

In an example, the ligands of the present invention can be released from endosomes in cells at specific locations in the skin of a subject. For example, molecules that promote an immune response (e.g. cytokines, adhesion molecules, co-stimulatory molecules, etc.). For example, antisense oligonucleotides designed to reduce the expression of ICAM-1 can be used to treat various inflammatory skin disorders. In particular, the expression of ICAM-1 has been associated such inflammatory skin disorders as allergic contact dermatitis, fixed drug eruption, lichen planus and psoriasis (Ho et al. 1990. J. Am. Acad. Dermatol., 22:64; Griffiths et al. 1989. Am. J. Pathology, 135:1045; Lisby et al. 1989. J. Dermatol. 120:479; Shiohara et al. 1989. Arch. Dermatol., 125:1371). By shining light on the location of the inflammatory skin disorder, the oligonucleotides of the present invention can also be released at the precise location of the skin inflammation. For instance, ICAM-1 antisense oligonucleotides released at the site of allergic contact dermatitis would act to reduce the expression of ICAM-1, thereby treating the allergic contact dermatitis.

The released ligand of the present invention may interact with an intracellular target (.e.g. to modulate the binding or interaction of two binding partners). The released ligand may act as an agonist to an activity of a molecule inside the cell.). The released ligand may act as an antagonist to an activity of a molecule inside the cell (e.g. a dominant negative mutant).

In addition, the ligands of the present invention can also be released from endosomes in cells at locations internal to the body of the subject using a flexible light source, thereby treating diseases or ailments that involve localized sites in organs or regions internal to the subject. In particular, ICAM-1 expression has been detected in the synovium of patients with rheumatoid arthritis (Hale et al. 1989. Arth. Rheum., 32:22), in pancreatic B-cells in diabetes (Campbell et al. 1989. Proc. Natl. Acad. Sci. U.S.A. 86:4282) and in thyroid follicular cells in patients with Graves' disease (Weetman et al. 1989. J. Endocrinol. 122:185), and has been associated with renal and liver allograft rejection (Faull et al. 1989. Transplantation 48:226). By using a flexible light source in a variant of endoscopy (see discussion above), it is possible to shine a light on the cells in the synovium of patients with rheumatoid arthritis or cells in the pancreas of subjects with diabetes. For instance, by shining a flexible light on the synovium of patients with rheumatoid arthritis, it is possible to activate the fluorophore associated with or attached to ICAM-1 antisense oligonucleotides, thereby causing the release of ICAM-1 antisense oligonucleotides from the endosomes of synovial cells. The ICAM-1 antisense oligonucleotides then act to block the expression of ICAM-1 in the synovium, thereby helping to alleviate the symptoms of rheumatoid arthritis.

In one embodiment, surgery can be performed so that the light source can be made to reach internal locations to activate the fluorescent fluorophore and cause the release of the oligonucleotides of the present invention from endosomes.

### Treatment of Neoplasia or Cancer

The subject invention is particularly well suited for the treatment of neoplasia. In particular, the ligands of the present invention include anti-sense oligonucleotides that act to reduce the expression of an oncogene. Further, several anti-sense oligonucleotides may be used in combination to inhibit the expression of several oncogenes that contribute to a cancerous phenotype.

“Neoplasia” or “neoplastic transformation” is the pathologic process that results in the formation and growth of a neoplasm, tissue mass, or tumor. Such process includes uncontrolled cell growth, including either benign or malignant tumors. Neoplasms include abnormal masses of tissue, the growth of which exceeds and is uncoordinated with that of the normal tissues and persists in the same excessive manner after cessation of the stimuli which evoked the change. Neoplasms may show a partial or complete lack of structural organization and functional coordination with the normal tissue, and usually form a distinct mass of tissue. One cause of neoplasia is dysregulation of the cell cycle machinery.

Neoplasms tend to grow and function somewhat independently of the homeostatic mechanisms which control normal tissue growth and function. However, some neoplasms remain under the control of the homeostatic mechanisms which control normal tissue growth and function. For example, some neoplasms are estrogen sensitive and can be arrested by anti-estrogen therapy. Neoplasms can range in size from less than 1 cm to over 6 inches in diameter. A neoplasm even 1 cm in diameter can cause biliary obstructions and jaundice if it arises in and obstructs the ampulla of Vater.

Neoplasms tend to morphologically and functionally resemble the tissue from which they originated. For example, neoplasms arising within he islet tissue of the pancreas resemble the islet tissue, contain secretory granules, and secrete insulin.

Clinical features of a neoplasm may result from the function of the tissue from which it originated. For example, excessive amounts of insulin can be produced by islet cell neoplasms resulting in hypoglycemia which, in turn, results in headaches and dizziness.

However, some neoplasms show little morphological or functional resemblance to the tissue from which they originated. Some neoplasms result in such non-specific systemic effects as cachexia, increased susceptibility to infection, and fever.

By assessing the histologic and others features of a neoplasm, it can be determined whether the neoplasm is benign or malignant. Invasion and metastasis (the spread of the neoplasm to distant sites) are definitive attributes of malignancy. Despite the fact that benign neoplasms may attain enormous size, they remain discrete and distinct from the adjacent non-neoplastic tissue. Benign tumors are generally well circumscribed and round, have a capsule, and have a grey or white color, and a uniform texture. By contrast, malignant tumor generally have fingerlike projections, irregular margins, are not circumscribed, and have a variable color and texture. Benign tumors grow by pushing on adjacent tissue as they grow. As the benign tumor enlarges it compresses adjacent tissue, sometimes causing atrophy. The junction between a benign tumor and surrounding tissue may be converted to a fibrous connective tissue capsule allowing for easy surgical remove of benign tumors. By contrast, malignant tumors are locally invasive and grow into the adjacent tissues usually giving rise to irregular margins that are not encapsulated making it necessary to remove a wide margin of normal tissue for the surgical removal of malignant tumors. Benign neoplasms tends to grow more slowly than malignant tumors. Benign neoplasms also tend to be less autonomous than malignant tumors. Benign neoplasms tend to closely histologically resemble the tissue from which they originated. More high differentiated cancers, cancers that resemble the tissue from which they originated, tend to have a better prognosis than poorly differentiated cancers. Malignant tumors are more likely than benign tumors to have an aberrant function (i.e. the secretion of abnormal or excessive quantities of hormones).

The histological features of cancer are summarized by the term “anaplasia.” Malignant neoplasms often contain numerous mitotic cells. These cells are typically abnormal. Such mitotic aberrations account for some of the karyotypic abnormalities found in most cancers. Bizarre multinucleated cells are also seen in some cancers, especially those which are highly anaplastic. “Dyplasia” refers to a pre-malignant state in which a tissue demonstrates histologic and cytologic features intermediate between normal and anaplastic. Dysplasia is often reversible.

“Anaplasia” refers to the histological features of cancer. These features include derangement of the normal tissue architecture, the crowding of cells, lack of cellular orientation termed dyspolarity, cellular heterogeneity in size and shape termed “pleomorphism.” The cytologic features of anaplasia include an increased nuclear-cytoplasmic ratio (nuclear-cytoplasmic ratio can be over 50% for malignant cells), nuclear pleomorphism, clumping of the nuclear chromatin along the nuclear membrane, increased staining of the nuclear chromatin, simplified endoplasmic reticulum, increased free ribosomes, pleomorphism of mitochondria, decrease in size and number of organelles, enlarged and increased numbers of nucleoli, and sometimes the presence of intermediate filaments.

As used herein, the term “cancer” includes a malignancy characterized by deregulated or uncontrolled cell growth, for instance carcinomas, sarcomas, leukemias, and lymphomas. The term “cancer” includes primary malignant tumors (e.g., those whose cells have not migrated to sites in the subject's body other than the site of the original tumor) and secondary malignant tumors (e.g., those arising from metastasis, the migration of tumor cells to secondary sites that are different from the site of the original tumor).

The term “carcinoma” includes malignancies of epithelial or endocrine tissues, including respiratory system carcinomas, gastrointestinal system carcinomas, genitourinary system carcinomas, testicular carcinomas, breast carcinomas, prostate carcinomas, endocrine system carcinomas, melanomas, choriocarcinoma, and carcinomas of the cervix, lung, head and neck, colon, and ovary. The term “carcinoma” also includes carcinosarcomas, which include malignant tumors composed of carcinomatous and sarcomatous tissues. An “adenocarcinoma” refers to a carcinoma derived from glandular tissue or a tumor in which the tumor cells form recognizable glandular structures.

The term “sarcoma” includes malignant tumors of mesodermal connective tissue, e.g., tumors of bone, fat, and cartilage.

The terms “leukemia” and “lymphoma” include malignancies of the hematopoietic cells of the bone marrow. Leukemias tend to proliferate as single cells, whereas lymphomas tend to proliferate as solid tumor masses. Examples of leukemias include acute myeloid leukemia (AML), acute promyelocytic leukemia, chronic myelogenous leukemia, mixed-lineage leukemia, acute monoblastic leukemia, acute lymphoblastic leukemia, acute non-lymphoblastic leukemia, blastic mantle cell leukemia, myelodyplastic syndrome, T cell leukemia, B cell leukemia, and chronic lymphocytic leukemia. Examples of lymphomas include Hodgkin's disease, non-Hodgkin's lymphoma, B cell lymphoma, epitheliotropic lymphoma, composite lymphoma, anaplastic large cell lymphoma, gastric and non-gastric mucosa-associated lymphoid tissue lymphoma, lymphoproliferative disease, T cell lymphoma, Burkitt's lymphoma, mantle cell lymphoma, diffuse large cell lymphoma, lymphoplasmacytoid lymphoma, and multiple myeloma.

For example, the therapeutic methods of the present invention can be applied to cancerous cells of mesenchymal origin, such as those producing sarcomas (e.g., fibrosarcoma, myxosarcoma, liosarcoma, chondrosarcoma, osteogenic sarcoma or chordosarcoma, angiosarcoma, endotheliosarcoma, lympangiosarcoma, synoviosarcoma or mesothelisosarcoma); leukemias and lymphomas such as granulocytic leukemia, monocytic leukemia, lymphocytic leukemia, malignant lymphoma, plasmocytoma, reticulum cell sarcoma, or Hodgkin's disease; sarcomas such as leiomysarcoma or rhabdomysarcoma, tumors of epithelial origin such as squamous cell carcinoma, basal cell carcinoma, sweat gland carcinoma, sebaceous gland carcinoma, adenocarcinoma, papillary carcinoma, papillary adenocarcinoma, cystadenocarcinoma, medullary carcinoma, undifferentiated carcinoma, bronchogenic carcinoma, melanoma, renal cell carcinoma, hepatoma-liver cell carcinoma, bile duct carcinoma, cholangiocarcinoma, papillary carcinoma, transitional cell carcinoma, chorioaencinoma, semonoma, or embryonal carcinoma; and tumors of the nervous system including gioma, menigoma, medulloblastoma, schwannoma or epidymoma. Additional cell types amenable to treatment according to the methods described herein include those giving rise to mammary carcinomas, gastrointestinal carcinoma, such as colonic carcinomas, bladder carcinoma, prostate carcinoma, and squamous cell carcinoma of the neck and head region. Examples of cancers amenable to treatment according to the methods described herein include vaginal, cervical, and breast cancers. One skilled in the art would readily recognize numerous oncogenes the expression of which can be reduced by anti-sense methods. Examples of oncogenes include, but are not limited to, c-Sis (encodes the PDGF B chain); int-2 (encodes a FGF-related growth factor); KGF (encodes an FGF-related growth factor); c-Fms (encodes colony stimulating factor-1 receptor); Flg (encodes a form of the FGF receptor); Neu (encodes an EGF-related receptor); Trk (including TrkA, TrkB, and TrkC) (encodes NGF receptor-like proteins); Met (encodes the hepatocyte growth factor/scatter factor receptor); c-Kit (encodes the mast cell growth factor receptor); v-src (encodes a protein tyrosin kinase); c-Src; Lck; Mas (encode the angiotensin receptor); c-Ras; Raf; Myc; Fos; and Jun. Oligonucleotides can readily be designed to inhibit the expression of any of these oncogenes.

One skilled in the art would readily recognize numerous tumor suppressors the expression of which can be increased by sense oligonucleotide methods. Examples of tumor suppressor genes include, but are not limited to RB1 (retinoblastoma susceptibility gene); p73, BRCA-1, BRCA-2, NF-1 (neurofibromatosis type-1), APC or FAP (familial adenomatosis polyposis coli), TGF-β, NF-2, merlin or NF2, VHL (Von Hipple-Lindau), WT-1 (Wilms tumor gene), bcl-2, bax, bad, bcl-xS, p16, p21, p27, p53, PTEN, Maspin, Uteroglogin, DCC (deleted in colon carcinoma), TSC1, TSC2, DPC4 or Smad4, MSH2, MLH1, VHL, CDKN2A, PTCH, and MEN1.

Because of the requirement that the cells to be treated must be exposed to light, the subject invention is particularly well suited to the treatment of melanomas (cancers derived from pigment cells in the skin) and carcinomas (cancers arising from epithelial cells) including bladder carcinomas, colon carcinomas, and gastrointestinal carcinomas. These cancers can be readily reached by a light source directed at the skin. Preferably, the light source is directed at the site of a tumor or sites where the cancer is likely to metastasize to. This invention can also be readily used in the treatment of adenocarcinomas (malignant glandular tumors). Various glands can be reached using a flexible endoscopic light source that can be directed at the gland containing the cancer. Preferably, the light source is directed specifically at a tumor site.

Administration of Ligands

Cells can be contacted with a single type of oligonucleotide, e.g., an antisense oligonucleotide specific for a single target molecule or with multiple oligonucleotides specific for multiple target genes. Alternatively, the media containing ligands of the present invention may include multiple types of ligands. A mixture of ligands may be used to inhibit the expression of several proteins that all contribute to a disorder or a disease state. For instance, several oncogenes may act together to contribute to the deregulation of proliferation and, therefore, a mixture of several ligands each inhibiting a different oncogene may be used together in the invention.

The ligands of the invention can be used in a variety of in vitro and in vitro situations to specifically degrade a target mRNA molecule. The instant methods and compositions are suitable for both in vitro and in vivo use.

In one embodiment, the oligonucleotides of the invention can be used to inhibit gene function in vitro in a method for identifying the functions of genes. The transcription genes that are identified, but for which no function has yet been shown can be inhibited to determine how the phenotype of a cell is changed when the gene is not transcribed. Such methods are useful for the validation of target genes for clinical treatment with antisense oligonucleotides or with other therapies.

In one embodiment, in vitro treatment of cells with ligands can be used for ex vivo therapy of cells removed from a subject (e.g., for treatment of leukemia or viral infection) or for treatment of cells which did not originate in the subject, but are to be administered to the subject (e.g., to eliminate transplantation antigen expression on cells to be transplanted into a subject). In addition, in vitro treatment of cells can be used in non-therapeutic settings, e.g., to study gene regulation and protein synthesis or to evaluate improvements made to oligonucleotides designed to modulate gene expression and/or protein synthesis.

In vivo treatment of cells can be useful in certain clinical settings where it is desirable to inhibit the expression of a protein. There are numerous medical conditions for which antisense therapy is reported to be suitable (see e.g., U.S. Pat. No. 5,830,653) as well as respiratory syncytial virus infection (WO 95/22553) influenza virus (WO 94/23028), and malignancies (WO 94/08003). Other examples of clinical uses of antisense oligonucleotides are reviewed, e.g., in Glaser. 1996. Genetic Engineering News 16:1. Exemplary targets for cleavage by antisense oligonucleotides include e.g., protein kinase Ca, ICAM-1, c-raf kinase, c-myb, and the bcr/abl fusion gene found in chronic myelogenous leukemia. Exemplary sense oligonucleotides include those encoding therapeutically relevant proteins. “Therapeutically relevant proteins” includes a protein that can be used in the treatment of a subject where the expression of a protein would be of benefit, e.g., in ameliorating the symptoms of a disease or disorder. For example, a therapeutically relevant protein can replace or augment protein expression in a cell which does not normally express a protein or which misexpresses a protein, e.g., a therapeutically relevant protein can compensate for a mutation by supplying a desirable protein. In addition, a “therapeutically relevant protein” can produce a beneficial outcome in a subject, e.g., can be used to produce a protein which vaccinates a subject against an infectious disease.

The optimal course of administration of the ligands may vary depending upon the desired result or on the subject to be treated. As used herein “administration” refers to contacting cells with ligands. The dosage of ligands may be adjusted to optimally reduce expression of a protein translated from a target mRNA, e.g., as measured by a readout of RNA stability or by a therapeutic response, without undue experimentation. For example, expression of the protein encoded by the nucleic acid target can be measured to determine whether or not the dosage regimen needs to be adjusted accordingly. In addition, an increase or decrease in RNA and/or protein levels in a cell or produced by a cell can be measured using any art recognized technique. By determining whether transcription has been decreased, the effectiveness of the oligonucleotide in inducing the cleavage of the target RNA can be determined.

As used herein, “pharmaceutically acceptable carrier” includes appropriate solvents, dispersion media, coatings, antibacterial and antifungal agents, isotonic and absorption delaying agents, and the like. The use of such media and agents for pharmaceutical active substances is well known in the art. Except insofar as any conventional media or agent is incompatible with the active ingredient, it can be used in the therapeutic compositions. Supplementary active ingredients can also be incorporated into the compositions.

Oligonucleotides may be incorporated into liposomes or liposomes modified with polyethylene glycol or admixed with cationic lipids for parenteral administration. Incorporation of additional substances into the liposome, for example, antibodies reactive against membrane proteins found on specific target cells, can help target the oligonucleotides to specific cell types.

Moreover, the present invention provides for administering the subject oligonucleotides with an osmotic pump providing continuous infusion of such oligonucleotides, for example, as described in Rataiczak et al. (1992 Proc. Natl. Acad. Sci. USA 89:11823-11827). Such osmotic pumps are commercially available, e.g., from Alzet Inc. (Palo Alto, Calif.). Topical administration and parenteral administration in a cationic lipid carrier are preferred.

With respect to in vivo applications, the formulations of the present invention can be administered to a patient in a variety of forms adapted to the chosen route of administration, namely, parenterally, orally, or intraperitoneally. Parenteral administration, which is preferred, includes administration by the following routes: intravenous; intramuscular; interstitially; intraarterially; subcutaneous; intra ocular; intrasynovial; trans epithelial, including transdermal; pulmonary via inhalation; ophthalmic; sublingual and buccal; topically, including ophthalmic; dermal; ocular; rectal; and nasal inhalation via insufflation. Intravenous administration is preferred among the routes of parenteral administration.

Pharmaceutical preparations for parenteral administration include aqueous solutions of the active compounds in water-soluble or water-dispersible form. In addition, suspensions of the active compounds as appropriate oily injection suspensions may be administered. Suitable lipophilic solvents or vehicles include fatty oils, for example, sesame oil, or synthetic fatty acid esters, for example, ethyl oleate or triglycerides. Aqueous injection suspensions may contain substances which increase the viscosity of the suspension include, for example, sodium carboxymethyl cellulose, sorbitol, and/or dextran, optionally, the suspension may also contain stabilizers. The ligands of the invention can be formulated in liquid solutions, preferably in physiologically compatible buffers such as Hank's solution or Ringer's solution. In addition, the oligonucleotides may be formulated in solid form and redissolved or suspended immediately prior to use. Lyophilized forms are also included in the invention.

Pharmaceutical preparations for topical administration include transdermal patches, ointments, lotions, creams, gels, drops, sprays, suppositories; liquids and powders. In addition, conventional pharmaceutical carriers, aqueous, powder or oily bases, and/or thickeners may be used in pharmaceutical preparations for topical administration.

Pharmaceutical preparations for oral administration include powders or granules, suspensions or solutions in water or non-aqueous media, capsules, sachets or tablets. In addition, thickeners, flavoring agents, diluents, emulsifiers, dispersing aids, and/or binders may be used in pharmaceutical preparations for oral administration. For transmucosal or transdermal administration, penetrants appropriate to the barrier to be permeated are used in the formulation. Such penetrants are known in the art, and include, for example, for transmucosal administration bile salts and fusidic acid derivatives, and detergents. Transmucosal administration may be through nasal sprays or using suppositories. For oral administration, the ligands are formulated into conventional oral administration forms such as capsules, tablets, and tonics. For topical administration, the ligands of the invention are formulated into ointments, salves, gels, or creams as known in the art.

Drug delivery vehicles can be chosen e.g., for in vitro, for systemic, or for topical administration. These vehicles can be designed to serve as a slow release reservoir or to deliver their contents directly to the target cell. An advantage of using some direct delivery drug vehicles is that multiple molecules are delivered per uptake. Such vehicles have been shown to increase the circulation half-life of drugs that would otherwise be rapidly cleared from the blood stream. Some examples of such specialized drug delivery vehicles which fall into this category are liposomes, hydrogels, cyclodextrins, biodegradable nanocapsules, and bioadhesive microspheres.

The described ligands may be administered systemically to a subject. Systemic absorption refers to the entry of drugs into the blood stream followed by distribution throughout the entire body. Administration routes which lead to systemic absorption include: intravenous, subcutaneous, intraperitoneal, and intranasal. Each of these administration routes delivers the ligand to accessible diseased cells. Following subcutaneous administration, the therapeutic agent drains into local lymph nodes and proceeds through the lymphatic network into the circulation. The rate of entry into the circulation has been shown to be a function of molecular weight or size. The use of a liposome or other drug carrier localizes the oligonucleotide at the lymph node. The oligonucleotide can be modified to diffuse into the cell, or the liposome can directly participate in the delivery of either the unmodified or modified oligonucleotide into the cell.

The chosen method of delivery will result in entry into cells. Exemplary delivery methods include liposomes (10-400 nm), hydrogels, controlled-release polymers, and other pharmaceutically applicable vehicles, and microinjection or electroporation (for ex vivo treatments).

The pharmaceutical preparations of the present invention may be prepared and formulated as emulsions. Emulsions are usually heterogenous systems of one liquid dispersed in another in the form of droplets usually exceeding 0.1 μm in diameter. The emulsions of the present invention may contain excipients such as emulsifiers, stabilizers, dyes, fats, oils, waxes, fatty acids, fatty alcohols, fatty esters, humectants, hydrophilic colloids, preservatives, and anti-oxidants may also be present in emulsions as needed. These excipients may be present as a solution in either the aqueous phase, oily phase or itself as a separate phase. Examples of naturally occurring emulsifiers that may be used in emulsion formulations of the present invention include lanolin, beeswax, phosphatides, lecithin and acacia. Finely divided solids have also been used as good emulsifiers especially in combination with surfactants and in viscous preparations. Examples of finely divided solids that may be used as emulsifiers include polar inorganic solids, such as heavy metal hydroxides, nonswelling clays such as bentonite, attapulgite, hectorite, kaolin, montmorillonite, colloidal aluminum silicate and colloidal magnesium aluminum silicate, pigments and nonpolar solids such as carbon or glyceryl tristearate.

Examples of preservatives that may be included in the emulsion formulations include methyl paraben, propyl paraben, quaternary ammonium salts, benzalkonium chloride, esters of p-hydroxybenzoic acid, and boric acid. Examples of antioxidants that may be included in the emulsion formulations include free radical scavengers such as tocopherols, alkyl gallates, butylated hydroxyanisole, butylated hydroxytoluene, or reducing agents such as ascorbic acid and sodium metabisulfite, and antioxidant synergists such as citric acid, tartaric acid, and lecithin.

In an embodiment, the compositions of ligands are formulated as microemulsions. A microemulsion is a system of water, oil and amphiphile which is a single optically isotropic and thermodynamically stable liquid solution. Typically microemulsions are prepared by first dispersing an oil in an aqueous surfactant solution and then adding a sufficient amount of a 4th component, generally an intermediate chain-length alcohol to form a transparent system.

Surfactants that may be used in the preparation of microemulsions include, but are not limited to, ionic surfactants, non-ionic surfactants, Brij 96, polyoxyethylene oleyl ethers, polyglycerol fatty acid esters, tetraglycerol monolaurate (ML310), tetraglycerol monooleate (MO310), hexaglycerol monooleate (PO310), hexaglycerol pentaoleate (PO500), decaglycerol monocaprate (MCA750), decaglycerol monooleate (MO750), decaglycerol sequioleate (S0750), decaglycerol decaoleate (DA0750), alone or in combination with cosurfactants. The cosurfactant, usually a short-chain alcohol such as ethanol, 1-propanol, or 1-butanol, serves to increase the interfacial fluidity by penetrating into the surfactant film and consequently creating a disordered film because of the void space generated among surfactant molecules. Microemulsions may, however, be prepared without the use of cosurfactants and alcohol-free self-emulsifying microemulsion systems are known in the art. The aqueous phase may typically be, but is not limited to, water, an aqueous solution of the drug, glycerol, PEG300, PEG400, polyglycerols, propylene glycols, and derivatives of ethylene glycol. The oil phase may include, but is not limited to, materials such as Captex 300, Captex 355, Capmul MCM, fatty acid esters, medium chain (C8-C12) mono-, di-, and tri-glycerides, polyoxyethylated glyceryl fatty acid esters, fatty alcohols, polyglycolized glycerides, saturated-polyglycolized C8-C10 glycerides, vegetable oils and silicone oil. Microemulsions are particularly of interest from the standpoint of drug solubilization and the enhanced absorption of drugs. Lipid based microemulsions (both oil/water and water/oil) have been proposed to enhance the oral bioavailability of drugs Microemulsions offer improved drug solubilization, protection of drug from enzymatic hydrolysis, possible enhancement of drug absorption due to surfactant-induced alterations in membrane fluidity and permeability, ease of preparation, ease of oral administration over solid dosage forms, improved clinical potency, and decreased toxicity (Constantinides et al. 1994. Pharmaceutical Research 11:1385; Ho et al. 1996. J. Pharm. Sci. 85:138-143). Microemulsions have also been effective in the transdermal delivery of active components in both cosmetic and pharmaceutical applications. It is expected that the microemulsion compositions and formulations of the present invention will facilitate the increased systemic absorption of ligands from the gastrointestinal tract, as well as improve the local cellular uptake of ligands within the gastrointestinal tract, vagina, buccal cavity and other areas of administration.

In one embodiment, the present invention employs various penetration enhancers to effect the efficient delivery of nucleic acids, particularly oligonucleotides, to the skin of animals. Even non-lipophilic drugs may cross cell membranes if the membrane to be crossed is treated with a penetration enhancer. In addition to increasing the diffusion of non-lipophilic drugs across cell membranes, penetration enhancers also act to enhance the permeability of lipophilic drugs.

Five categories of penetration enhancers that may be used in the present invention include: surfactants, fatty acids, bile salts, chelating agents, and non-chelating non-surfactants Other agents may be utilized to enhance the penetration of the administered oligonucleotides include: glycols such as ethylene glycol and propylene glycol, pyrrols such as 2-15 pyrrol, azones, and terpenes such as limonene and menthone.

The oligonucleotides, especially in lipid formulations, can also be administered by coating a medical device, for example, a catheter, such as an angioplasty balloon catheter, with a cationic lipid formulation. Coating may be achieved, for example, by dipping the medical device into a lipid formulation or a mixture of a lipid formulation and a suitable solvent, for example, an aqueous-based buffer, an aqueous solvent, ethanol, methylene chloride, chloroform and the like. An amount of the formulation will naturally adhere to the surface of the device which is subsequently administered to a patient, as appropriate. Alternatively, a lyophilized mixture of a lipid formulation may be specifically bound to the surface of the device. Such binding techniques are described, for example, in K. Ishihara et al., Journal of Biomedical Materials Research, Vol. 27, pp. 1309-1314 (1993), the disclosures of which are incorporated herein by reference in their entirety.

The useful dosage to be administered and the particular mode of administration will vary depending upon such factors as the cell type, or for in vivo use, the age, weight and the particular animal and region thereof to be treated, the particular ligand and delivery method used, the therapeutic or diagnostic use contemplated, and the form of the formulation, for example, suspension, emulsion, micelle or liposome, as will be readily apparent to those skilled in the art. Typically, dosage is administered at lower levels and increased until the desired effect is achieved. When lipids are used to deliver the oligonucleotides, the amount of lipid compound that is administered can vary and generally depends upon the amount of oligonucleotide agent being administered. For example, the weight ratio of lipid compound to oligonucleotide agent is preferably from about 1:1 to about 15:1, with a weight ratio of about 5:1 to about 10:1 being more preferred. Generally, the amount of cationic lipid compound which is administered will vary from between about 0.1 milligram (mg) to about 1 gram (g). By way of general guidance, typically between about 0.1 mg and about 10 mg of the particular oligonucleotide agent, and about 1 mg to about 100 mg of the lipid compositions, each per kilogram of patient body weight, is administered, although higher and lower amounts can be used.

The agents of the invention are administered to subjects or contacted with cells in a biologically compatible form suitable for pharmaceutical administration. By “biologically compatible form suitable for administration” is meant that the ligand is administered in a form in which any toxic effects are outweighed by the therapeutic effects of the ligand. In one embodiment, ligands can be administered to subjects. The term subject is intended to include living organisms, e.g., prokaryotes and eukaryotes. Examples of subjects include mammals, e.g., humans, dogs, cats, mice, rabbits, rats, and transgenic non-human animals. Most preferably the subject is a human.

Administration of an active amount of a ligand of the present invention is defined as an amount effective, at dosages and for periods of time necessary to achieve the desired result. For example, an active amount of a ligand may vary according to factors such as the type of cell, the ligand used, and for in vivo uses the disease state, age, sex, and weight of the individual, and the ability of the ligand to elicit a desired response in the individual. Establishment of therapeutic levels of ligands within the cell is dependent upon the rates of uptake and efflux degradation. Decreasing the degree of degradation prolongs the intracellular half-life of the ligand. Thus, chemically-modified oligonucleotides, e.g., with modification of the phosphate backbone, may require different dosing.

The exact dosage of a ligand and number of doses administered will depend upon the data generated experimentally and in clinical trials. Several factors such as the desired effect, the delivery vehicle, disease indication, and the route of administration, will affect the dosage. The expected in vivo dosage is between about 0.001-200 mg/kg of body weight/day. For example, the oligonucleotides can be provided in a therapeutically effective amount of about 0.1 mg to about 100 mg per kg of body weight per day, and preferably of about 0.1 mg to about 10 mg per kg of body weight per day, to bind to a nucleic acid in accordance with the methods of this invention. Dosages can be readily determined by one of ordinary skill in the art and formulated into the subject pharmaceutical compositions. Preferably, the duration of treatment will extend at least through the course of the disease symptoms.

Dosage regimen may be adjusted to provide the optimum therapeutic response. For example, the ligand may be repeatedly administered, e.g., several doses may be administered daily or the dose may be proportionally reduced as indicated by the exigencies of the therapeutic situation. One of ordinary skill in the art will readily be able to determine appropriate doses and schedules of administration of the subject ligands, whether the ligands are to be administered to cells or to subjects.

The practice of the present invention will employ, unless otherwise indicated, conventional techniques of cell biology, cell culture, molecular biology, microbiology, recombinant DNA, and immunology, which are within the skill of the art. Such techniques are explained fully in the literature. See, for example, Molecular Cloning A Laboratory Manual, 2nd Ed., ed. by Sambrook, J. et al. (Cold Spring Harbor Laboratory Press (1989)); Short Protocols in Molecular Biology, 3rd Ed., ed. by Ausubel, F. et al. (Wiley, NY (1995)); DNA Cloning, Volumes I and II (D. N. Glover ed., 1985); Oligonucleotide Synthesis (M. J. Gait ed. (1984)); Mullis et al. U.S. Pat. No. 4,683,195; Nucleic Acid Hybridization (B. D. Hames & S. J. Higgins eds. (1984)); the treatise, Methods In Enzymology (Academic Press, Inc., N.Y.); Immunochemical Methods In Cell And Molecular Biology (Mayer and Walker, eds., Academic Press, London (1987)); Handbook Of Experimental Immunology, Volumes I-IV (D. M. Weir and C. C. Blackwell, eds. (1986)); and Miller, J. Experiments in Molecular Genetics (Cold Spring Harbor Press, Cold Spring Harbor, N.Y. (1972)).

The invention is further illustrated by the following examples, which should not be construed as further limiting. The contents of all references, pending patent applications and published patents, cited throughout this application are hereby expressly incorporated by reference.

### EXAMPLES

### Example 1

### Enhanced Availability of Ligand in A549 Cells

A549 cells were maintained in high glucose DMEM (Gibco-BRL) supplemented with 10% Fetal Bovine Serum (Gibco-BRL), 2 mM L-Glutamine (Gibco-BRL), and 1× penicillin/streptomycin (Gibco-BRL).

Seed cell suspensions were prepared by combining 75 μl of 1 mM oligomer (uniformly morpholino modified as taught, e.g., in Summerton, J. et al. Antisense Research and Development, Morpholino and Phosphorothioate Antisense Oligomers Compared in Cell-Free and In-Cell Systems 7:63-70 (1997)) per 425 μl cell suspension.

24-well plates were seeded with 0.5 ml 10-30K A549 cells per well. The cells are preferably evenly distributed across the plate. Cells were incubated for 18-24 hours at 37° C. in a humidified CO₂ incubator. The media was aspirated from the cells and each well rinsed with 0.5 ml of Opti-MEM reduced serum medium (GIBCO-BRL). The media was aspirated and 0.5 ml of Opti-MEM was added to each well a second time.

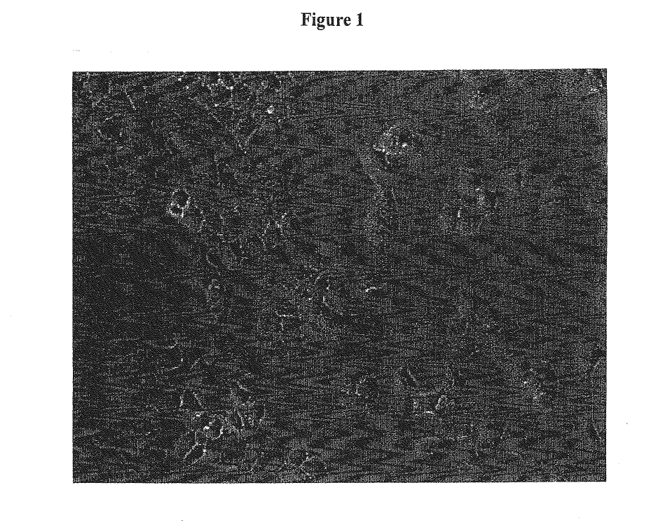

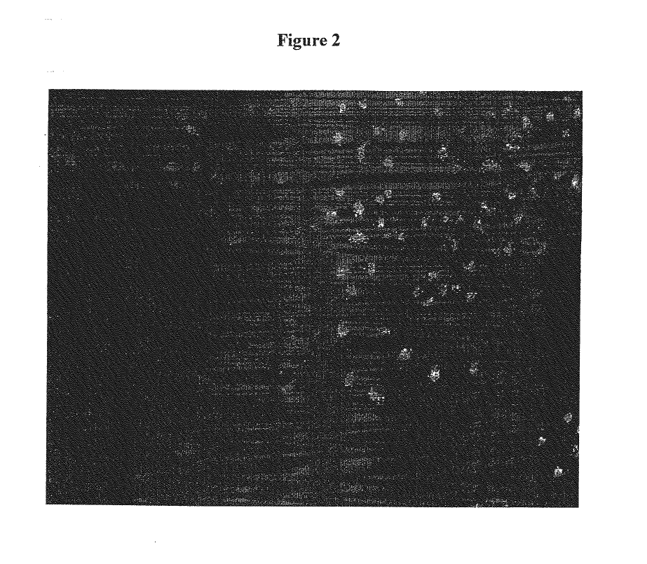

The cells were placed under light (450-490 nm) emitted from a 100 W mercury arc light source through a B-2E Epi-fluorescence filter for approximately 2 minutes, and uptake by fluorescence microscopy was evaluated. FIG. 1 is a bright field image showing the A549 cells. In FIG. 2, the fluorescent image shows the localization of the fluorescently tagged oligomer. The left side of the field has been illuminated with exication wavelength for approximately 2 minutes. While some photobleaching of the fluorphore has occurred, the staining has clearly moved in large part from the endosomes (punctate staining) to the whole cell (diffuse staining).

### Example 2

### Enhanced Availability of Ligand in HUVECs (Human Umbilical Vein Endothelial Cells)

Human umbilical vein endothelial cells (HUVECs) were maintained in HUVEC media: EBM (Clonetics) supplemented with 8% Fetal Bovine Serum (Gibco-BRL), 2 mM L-Glutamine (Gibco-BRL), and 1× penicillin/streptomycin (Gibco-BRL), hEGF, Hydrocortisone, GA-1000, BBE, and 2% FBS (Clonetics).

On the day before transfection 24-well plates were coated with 0.5 ml/well gelatin for 5 minutes. Gelatin was aspirated and plates seeded with 0.5 ml 10-30K HUVECs per well. (Cells are preferably about 70% confluent at the start of transfection, and should be evenly distributed across the plate.) The cells are incubated at 37° C. in a humidified CO₂ incubator.

On the day of transfection, a 300 μM stock solution of uniformly morpholino modified oligomer was prepared by diluting 300 μl of oligomer per 700 μl of HUVEC media. The media was aspirated from the cells, and 0.5 ml of the oligomer/HUVEC media was added to each well taking care not to let the cells dry out during the changing of media. The cells were incubated at 37° C. in a humidified CO₂ incubator.

After 48 hours of incubation, the media was aspirated from the cells, and replaced with 0.5 ml of fresh oligomer/HUVEC media, again taking care not to let the cells dry out during the changing of media.

The cells were incubated at 37° C. in a humidified CO₂ incubator. After 48 hours of incubation, the media was aspirated from the cells, and replaced with 0.5 ml of fresh oligomer/HUVEC media to each well. The cells were incubated at 37° C. in a humidified CO₂ incubator.

After 48 hours of incubation, the media was aspirated from the cells each well rinsed with 0.5 ml of Opti-MEM. The media was aspirated and 0.5 ml of Opti-MEM was added to each well a second time.

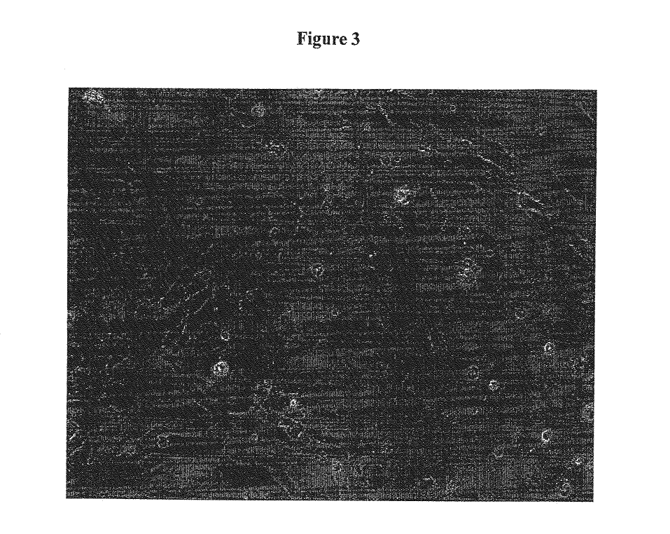

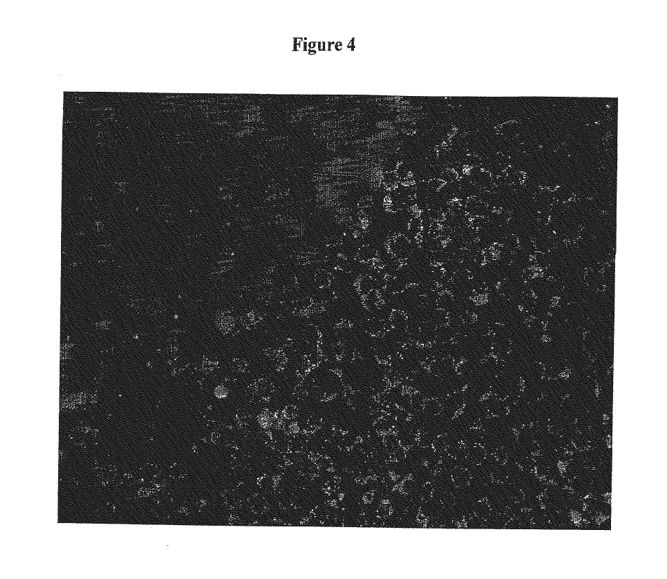

The cells were placed under light (450-490 nm) emitted from a 100 W mercury arc light source through a B-2E Epi-fluorescence filter for 2 minutes, and evaluate uptake by fluorescence microscopy. FIG. 3 is a bright field image showing the HUVEC cells. In FIG. 4, the fluorescent image shows the localization of the fluorescently tagged oligomer. The upper left side of the field has been illuminated with exication wavelength for approximately 2 minutes. While some photobleaching of the fluorphore has occurred, the staining has clearly moved in large part from the endosomes (punctate staining) to the whole cell (diffuse staining, upper left).

### Equivalents

Those skilled in the art will recognize, or be able to ascertain using no more than routine experimentation, many equivalents to the specific embodiments of the invention described herein. Such equivalents are intended to be encompassed by the following claims.

1. A method of delivering a ligand to a cell comprising:
a) contacting a cell with a ligand and a fluorophore; and
b) illuminating the cell with a light that activates the fluorophore such that the ligand is delivered to the cell.

2. The method of claim 1, wherein the ligand is an oligonucleotide.

3. The method of claim 1, wherein the ligand is peptide.

4. The method of claim 1, wherein ligand is a fluorescent virus.

5. The method of claim 1, wherein the ligand is a morpholino oligonucleotide.

6. The method of claim 1, wherein the ligand is a sense oligonucleotide.

7. The method of claim 1, wherein the ligand is an antisense oligonucleotide.

8. The method of claim 1, wherein the ligand enters an endosome of the cell during step (a).

9. The method of claim 8, wherein the illuminating of step (b) causes the endosome containing the ligand to release the ligand.

10. The method of claim 1, wherein the light has a wavelength of about 10 to about 380 nm.

11. The method of claim 1, wherein the light has a wavelength of about 380 to about 500 nm.

12. The method of claim 1, wherein the cells are illuminated for less than about 2 minutes.

13. The method of claim 1, wherein the cells are illuminated for less than about 1 minute.

14. The method of claim 1, wherein the light of step (b) is produced from a flexible endoscopic light source.

15. The method of claim 1, wherein the fluorophore and the ligand are linked via a covalent linkage.

16. The method of claim 1, wherein the fluorophore is a fluorescein fluorophore.

17. The method of claim 1, wherein the fluorophore and the ligand contacted with the cell simultaneously.

18-32. (canceled)

33. The method of claim 17, wherein the localized site is inside the colon of the subject.

34. The method of claim 17, wherein the localized site is on the skin of the subject.

35. The method of claim 17, wherein the localized site is a tumor.

36. A method of modulating protein production at a localized site in a subject comprising:
a) exposing a group of cells of the subject to a ligand and a fluorophore; and
b) illuminating the cells at a localized site in the subject with light that activates the fluorophore such that protein production at a localized site in the subject is modulated.

37. The method of claim 36, wherein protein production is enhanced.

38. The method of claim 37, wherein protein production is inhibited.

39. A method of modulating protein activity at a localized site in a subject comprising:
a) exposing a group of cells of the subject to a ligand and a fluorophore; and
b) illuminating the cells at a localized site in the subject with light such that the fluorophore is activated and protein activity at a localized site in a subject is modulated.

40. The method of claim 39, wherein protein activity is enhanced.

41. The method of claim 39, wherein protein activity is inhibited.

42. (canceled)
